(19) United States
(12) Patent Application Publication
(10) Pub. No.: US 2016/0038077 A1
(43) Pub. Date: Feb. 11, 2016

(54) Systems, Devices, and/or Methods for Identifying Time Periods of Insufficient Blood Glucose Testing

(71) Applicant: INSPARK TECHNOLOGIES, INC., Charlottesville, VA (US)

(72) Inventor: Erik A Otto, Charlottesville, VA (US)

(21) Appl. No.: 14/780,944

(22) PCT Filed: Jun. 13, 2014
(86) PCT No.: PCT/US14/42222

§ 371 Date: Sep. 28, 2015

Related U.S. Application Data
(60) Provisional application No. 61/840,418, filed on Jun. 27, 2013.

(51) Int. Cl. A61B 5/00; A61B 5/145

(52) U.S. Cl. CPC A61B 5/486; A61B 5/14532; A61B 5/742; A61B 5/7282; A61B 5/4833; A61B 5/7475; A61B 5/0022

(57) ABSTRACT

Certain exemplary embodiments provide devices, methods, systems, and/or computer program products related to the maintenance of optimal control of diabetes, directed to determining patterns of inadequate testing during periods that tend to precede periods of poor glycemic control, or that can be presented in the context of daily periods of poor glycemic control. A user can be notified of the pattern of insufficient testing in the time period immediate precedent to or during the period of insufficient testing, so that a detrimental pattern can be avoided and/or mitigated by the user through increased self-testing.

### CROSS-REFERENCES TO RELATED APPLICATIONS

This application claims priority to, and incorporates by reference herein in its entirety, pending U.S. Provisional Patent Application Ser. No. 61/840,418 (Attorney Docket No. 1048-003), filed 27 Jun. 2013.

### BRIEF DESCRIPTION OF THE DRAWINGS

A wide variety of potential practical and useful embodiments will be more readily understood through the following detailed description of certain exemplary embodiments, with reference to the accompanying exemplary drawings in which:

FIG. 1 is a flowchart of an exemplary embodiment of a method;

FIG. 2 is a flowchart of an exemplary embodiment of a method;

FIG. 3 is a flowchart of an exemplary embodiment of a method;

FIG. 4 is a flowchart of an exemplary embodiment of a method;

FIG. 5 is a display of an exemplary output;

FIG. 6 is a diagram of exemplary hardware and functional components of a system;

FIG. 7 is a table of exemplary statistical values;

FIG. 8 is a table of exemplary statistical values;

FIG. 9 is a table of exemplary statistical values;

FIG. 10 is a an exemplary embodiment of a diagram of glucose testing patterns; and

FIG. 11 is a block diagram of an exemplary embodiment of an information device 11000.

### DETAILED DESCRIPTION

Certain exemplary embodiments provide devices, methods, systems, and/or computer program products related to the maintenance of optimal control of diabetes, directed to determining patterns of inadequate testing during periods of poor glycemic control or that tend to precede periods of poor glycemic control. A user can be notified of the pattern of insufficient testing in the time period immediate precedent to or during the period of insufficient testing, so that a detrimental pattern can be avoided and/or mitigated by the user through increased self-testing. The devices, methods, systems, and/or computer program products pertain directly to the enhancement of diabetes management software and hardware, including software that resides on devices such as blood glucose measurement devices, telephonic systems, and/or networked computer systems.

Certain exemplary embodiments provide substantially real-time information to the user about upcoming periods of inadequate testing that may be causing poor glucose control, or leading to poor glucose control in future periods. This real-time information is directed to providing insight to the user such that the user can take appropriate action, such as increased testing in a designated time period, thereby leading to better glycemic control. Either during daily times that are preprogrammed, or during a self-monitoring of blood glucose (“SMBG”) measurement and prior to the presentation of SMBG result the device evaluates historical patterns of glycemia and, based on this evaluation, issues warnings about inadequate testing for future time periods either immediately in real-time, or at the time a glucose measurement is presented, or in a future time period proximal to the time period of inadequate testing.

The present patent application is related to U.S. patent application Ser. No. 11/943,226 and entitled “Systems, methods and computer program codes for recognition of patterns of hyperglycemia and hypoglycemia, increased glucose variability, and ineffective self-monitoring in diabetes”, the entire disclosure of which is hereby incorporated by reference in this application, and which shall be referred to herein as “Reference 1”.

The basic problem that people with diabetes have relates to the transfer of sugar, contained in the blood, across cell membranes. This in turn makes it difficult for the body to maintain sugar levels in the blood at the correct level. In the treatment of diabetes, patients regularly check blood glucose levels using a self-testing kit. By comparing the result of a self-test with the blood glucose level considered normal, a patient is able to estimate the amount of insulin that should be taken to keep the blood glucose level near normal, or what to eat to bring blood glucose into equilibrium, or how much to exercise. Too much blood sugar (e.g. due to the patient injecting too little insulin) or eating more than the prescribed amount is defined as hyperglycemic, while too little blood sugar (e.g. due to the patient injecting too much insulin) is defined as hypoglycemic. These are considered to be short-term complications of diabetes and can cause acute symptoms or be a factor in the development of long-term complications. Diabetic patients can also suffer problems arising from their condition that only become apparent in the longer term. These problems include retinopathy, peripheral neuropathy, nephropathy, and an increased risk of a number of cardiovascular events including stroke and myocardial infarction.

In order to monitor blood sugar levels, people with diabetes can perform SMBG periodically to obtain information about their blood glucose levels using a handheld blood glucose monitoring device. This information is used by patients and clinicians to guide treatment and management decisions so that blood glucose levels can be kept in an optimal range to avoid hypoglycemia and hyperglycemia.

Testing is performed several times a day by most patients on insulin, but even with frequent testing there can be a lack of information as glucose levels fluctuate significantly throughout the day. Moreover, patients might not have readily available information or analytical tools providing insight about what might be the best time of day to test, or what time of day there might be problematic patterns that warrant a greater frequency of monitoring.

Patients might be able to have better outcomes by obtaining information on their condition in real time, i.e., when they are making management decisions, so that the information is immediately actionable, without the need for patients to analyze detailed charts and graphs.

Continuous glucose monitoring (CGM) systems can provide frequent (every 1-20 minutes) feedback on blood glucose control levels in real-time, such as via an imbedded cannula and a portable device with display (in addition to a SMBG meter). CGM devices might not be as accurate as conventional SMBG meters, and can be subject to frequent calibration with an SMBG meter.

Certain exemplary embodiments provide analytical methods to evaluate both SMBG testing patterns and glucose control to provide insight to the user substantially in real time about advantageous testing times, so that patients can test more optimally throughout the day to improve their glucose control. Certain exemplary embodiments provide feedback to users about upcoming testing patterns in their day and their relation to deleterious blood sugar patterns. Certain exemplary embodiments push this information to the user via messaging in real time when they are actively managing their condition, either when they test or when they are on the go.

1. Binning of Blood Glucose Data for Evaluation

Certain exemplary embodiments identify periods of inadequate testing and deleterious glucose patterns via a sufficient database of blood glucose readings. The number of weeks of SMBG readings may be from as little as two days, or to over twelve weeks, but is preferably approximately two to eight weeks. Preferably, there are at least five readings per time period. The total number of SMBG readings may be from at least 30 readings, but preferably 60.

A day (i.e., a twenty-four hour period) may be divided into time bins with predetermined durations, from approximately two to approximately ten hours long. In accordance with Reference 1, for the purposes of clustering blood glucose data into time bins, preferred time periods of the day can be:

TABLE 1
Time Bins

| Time Bin | | | |
|---|---|---|---|
| Description | Duration | Time Boundaries | Bin Number |
| Night | 8 hours | 11:00 pm-6:59 am | 1 |
| Morning | 4 hours | 7:00 am to 10:59 am | 2 |
| Mid day | 4 hours | 11:00 am to 2:59 pm | 3 |
| Afternoon | 4 hours | 3:00 pm to 6:59 pm | 4 |
| Evening | 4 hours | 7:00 pm to 10:59 pm | 5 |

Time periods can also be specific to certain pre-prandial and post-prandial time periods, either preset in the system or designated by the user. Time periods for specific daily activities such as exercise, naps, work, or other can be pre-set or identified by the user as well.

2. Evaluation of Periods of Inadequate Testing:

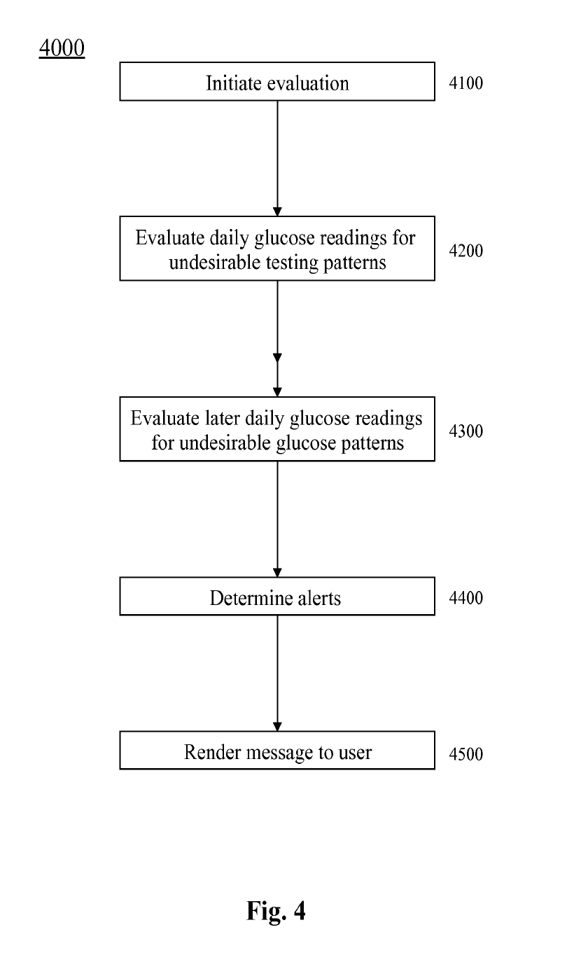

One exemplary method for identifying inadequate SMBG testing patterns, as disclosed in Reference 1, evaluates a percentage of blood glucose readings occurring in a time period relative to the rest of the patient's day. More specifically, if a time bin is found contains less than α%, or more than β% of a person's SMBG readings, then this time bin will be identified as a period of insufficient, or excessive testing, respectively. The parameters α% and β% can be set at any reasonable values, e.g. α%=5% and β%=50%. If one or both of these thresholds is exceeded, a message would be issued as presented in FIG. 4. The message would include a warning about insufficient testing if the testing frequency is below α%, and a warning about excessive testing is the testing frequency is higher than β%. Reference 1 also discloses that data deficient time periods also include those time periods where there is insufficient readings to generate pattern information. We shall call these criteria, as outlined by Reference 1, and when applied collectively, as “Criterion A” when α%=10%.

However the Reference 1 does not provide a method for indicating to the patient when there are pattern of deleterious glucose following a period of insufficient testing. The importance and utility of this novel method was discovered via the evaluation of a dataset of 436 patients with the characteristics below:

Dataset Characteristics
Type of Diabetes: Type1=146 (33%), Type 2=278 (64%), Missing=12 (3%)
Average glucose (AG): AG<154=107 (24%), AG>=154 and <183=145 (31%), AG>=183 and <212=110 (24%), AG>=212=94 (21%)
Daily Test Frequency: Average of 2-3/day=222 (49%), Average of 3-4/day=145 (32%), Average of 4-5/day=54 (12%), average of >5 per day=28(6%)

This dataset had 445340 evaluable time periods (i.e. daily periods of time with enough data to evaluate patterns according to a need for 60 readings in the last 30 days across all periods of time, and at least 5 readings in the last 30 days). Of these time periods, 116529 had a pattern identified of hyperglycemia, hypoglycemia, or variability. Of these time periods with any identified pattern, 17816 periods with patterns follow a data deficient time period (15% of the total). These deleterious patterns may not otherwise have been identified in a messaging paradigm as described in Reference 1 because patients would not be testing in the data deficient time bin, and thus the messaging system would not generate a message about them and the user may not be aware of them. In the disclosed embodiments, however, all of these time periods would have been identified and messaged to the user, and at the same time as a message about the test deficiency that precedes it. This latter point is important because there could be a causal effect of deficient testing on the manifestation of the deleterious pattern of glycemic control.

The utility of exemplary novel methods of messaging the patient about inadequate testing patterns can be demonstrated by the fact that these time periods following data deficient time periods have a greater propensity for deleterious glucose patterns. As shown in Table 2 below, In our evaluation of 436 patients with diabetes, 24.5% of non-data deficient time periods has at least one deleterious pattern of hyperglycemia, hypoglycemia, or glycemic variability; however, 25.5% of time periods following a data-deficient time period had a deleterious pattern of hyperglycemia, hypoglycemia and glycemic variability. Thus, on a relative basis there are 4% more deleterious glucose patterns following a period of deficient testing, and this proportional difference was statistically significant (p<0.001) lending credence to the utility of a messaging system that identifies these periods following a period of deficient testing.

TABLE 2
The relative number of deleterious patterns in a period following
a data deficient time period using test deficiency Criterion A.

| | Calculation | Value using |
|---|---|---|
| Desired Output | Variables | Criterion A |
| Total number of evaluable periods in | A | 402358 |
| dataset of 436 patients | | |
| Total number of evaluable periods | B | 70002 |
| that follow data deficient time | | |
| periods | | |
| Total number of periods with any | C | 116529 |
| deleterious hyper, hypo or variability | | |
| patterns | | |
| Total number of periods with any | D | 17816 |
| deleterious hyper, hypo or variability | | |
| patterns following data-deficient | | |
| time periods | | |
| Number of periods with any | C − D = E | 98713 |
| deleterious hyper, hypo or variability | | |
| patterns that do not follow data- | | |
| deficient time periods | | |
| % of total evaluable periods that | E/A = F | 24.5% |
| have deleterious patterns | | |
| % of evaluable periods following | D/B = G | 25.5% |
| data deficient time periods that have | | |
| deleterious patterns | | |
| Relative number of deleterious | G/F | 1.04 |
| patterns in periods following data | | |
| deficient time periods. | | |

As described above the evaluation shown in Table 2 was performed using evaluation criterion for deficient testing described in Reference 1 (Criterion A). However that testing criterion do not take into account the value of recent testing. For example, relevant testing for more than 15 days before the time bin is being evaluated might be available, but not in the most recent 15 days of the 30 day period, and thus the data deficiency determination may be irrelevant vis a vis the most proximal glucose readings and formation of patterns. We thus tested novel criterion of, in addition to applying Criterion A, also requiring at least half of the percent of the bin threshold in the most recent 15 days of readings (this shall be heretofore referred to as Criterion B). We also tested novel criterion of requiring at least half of the percent of the bin threshold in the most recent 10 days of testing (this shall be heretofore referred to as Criterion C). As can be seen in the following Table 3, this novel criterion increased the relative number of patterns in period following data deficient time periods number to 1.12 (p<0.001) and 1.37 (p<0.001) for Criterion B and Criterion C respectively, thus demonstrating that the additional data deficient time periods identified by these criteria tend to be followed by a significantly higher proportion of deleterious patterns than the rest of the time periods, as compared to Criterion A as shown in Table 2 (1.04).

TABLE 3
The relative number of deleterious patterns in a period following a data
deficient time period with novel data deficiency Criterion B and C.

| | Calculation | Value for | Value for |
|---|---|---|---|
| Desired Output | Variables | Criterion B | Criterion C |
| Total number of evaluable periods in | A | 402358 | 402358 |
| dataset of 436 patients | | | |
| Total number of evaluable periods | B | 88896 | 137107 |
| that follow data deficient time | | | |
| periods | | | |
| Total number of periods with any | C | 116529 | 116529 |
| deleterious hyper, hypo or variability | | | |
| patterns | | | |
| Total number of periods with any | D | 23125 | 37123 |
| deleterious hyper, hypo or variability | | | |
| patterns following data-deficient | | | |
| time periods | | | |
| Number of periods with any | C − D = E | 93404 | 79406 |
| deleterious hyper, hypo or variability | | | |
| patterns that do not follow data- | | | |
| deficient time periods | | | |
| % of total evaluable periods that | E/A = F | 23.2% | 19.7% |
| have deleterious patterns | | | |
| % of evaluable periods following | D/B = G | 26.0% | 27.1% |
| data deficient time periods that have | | | |
| deleterious patterns | | | |
| Relative number of deleterious | G/F | 1.12 | 1.37 |
| patterns in periods following data | | | |
| deficient time periods | | | |

3. Identification of Deleterious Glucose Patterns:

Exemplary preferred devices, systems, methods, and/or algorithms enabling the evaluation of deleterious glucose patterns in daily patterns are the subject of Reference 1, which is incorporated by reference into this application. The theoretical background has been established by the theory of risk analysis of blood glucose (“BG”) data therein, and follows previously developed and disclosed technology. Methods and algorithms have been first developed using general statistical assumptions for the deviation of glucose levels or testing patterns within a certain time period from the grand glucose mean or optimal pattern of a person. Then, the resulting algorithms were applied to a large data set (N=335 subjects) to validate the algorithms and to determine ranges for the algorithm parameters.

The algorithms identifying patterns of hyperglycemia and hypoglycemia can take the form of identifying a two or more blood glucose readings that exceed a given clinical threshold in the preceding days, or evaluating if the average blood glucose level in that time period over a predetermined time exceeds a predefined threshold. An exemplary preferred method of identifying these patterns, involves evaluating a composite probability of blood glucose levels exceeding a clinical threshold as well as a deviation from the rest of the day's time periods.

Algorithms for identifying glucose variability can take the form of a simple range, an interquartile range, a standard deviation, or percentage out of range. An exemplary preferred method evaluates a composite probability of variability exceeding a clinical threshold as well as a deviation from the rest of the day's time periods in that time bin.

More specifically, to identify these patterns certain exemplary embodiments work through several sequential steps described in detail below. To begin, a 24-hour daily SMBG profile of a person can be split into fixed periods of time with a predetermined duration, beginning at the time of a SMBG reading, or at another predetermined time. Then, based on historical SMBG data of approximately two weeks to approximately twelve weeks, the average glucose in each period of time can be evaluated for deviations towards hyperglycemia, hypoglycemia, or higher glucose variability. These deviations can be assessed at two levels for:
Exceeding absolute thresholds identified by population data, literature, and accepted clinical practice guidelines, and
Exceeding idiosyncratic thresholds, i.e. individual threshold, determined via analysis of the glycemic patterns of each individual. If any of these two conditions are present, the time period can be declared as a period of high risk for hyperglycemia or hypoglycemia. The judgment of each of the conditions can be governed by a pair of parameters for hyperglycemia and a pair of parameters for hypoglycemia, as follows:

For Patterns of Hyperglycemia:
BG threshold parameter (α1), reflecting population-level definition of high BG. For example, α1=180 mg/dl or α1=200 mg/dl can be acceptable values.
Individual composite probability threshold (β1) reflecting the idiosyncratic chance that BG would be high for this particular individual during this particular period of time. For example, β1=0.4 to 0.6 can be acceptable values.

For Patterns of Hypoglycemia:
BG threshold parameter (α2), reflecting population-level definition of low BG. For example, α2=70 mg/dl or α2=75 mg/dl are acceptable values.
Individual composite probability threshold (β2) reflecting the idiosyncratic chance that BG would be low for this particular individual during this particular period of time. For example, β=0.01 to 0.2 can be acceptable values. Steps of the Algorithms Identifying Patterns of Hyperglycemia and Hypoglycemia: the first seven steps of these two algorithms are identical:
For Patterns of Glucose Variability: As described in Reference 1, one example of thresholds that result in a favorable messaging frequency is an average daily risk range (“ADRR”) threshold (α) set at 40 and the deviation probability threshold (β) set at 0.6.
Thresholds of the Algorithms: The specific threshold values used by the algorithms can be determined by the manufacturer of the device using the algorithms, the clinician managing the patient using the algorithms, or the user, and can be based on the acceptability of the frequency of messages vs. utility of the messages.

Evaluation Steps:

(1) Retrieve all SMBG data collected during the last 2-12 weeks of monitoring together with the time of each reading.

(2) At the time of evaluation, split each 24-hour day into M time bins with predetermined duration (2-12 hours) in accordance with Table 1.

(3) Classify all SMBG readings into these time bins: Let Xₖ₁, Xₖ₂, . . . , X_{kNk} be the SMBG readings that fell into time bin k over the last 30 days of SMBG, e.g. k=1,2, . . . , M; where Nₖ=number of SMBG readings in bin k, and N=the number of SMBG readings in total in the several week time period under evaluation.

(4) For each time bin k compute mean (“ x̅”) and standard deviation (“SD”) of BG as follows:

$${\overline{X}}_{k} = \frac{1}{{N}_{k}} {\sum }_{k = 1}^{{N}_{k}} {X}_{ki}$$

$${SD}_{k}^{2} = \frac{1}{{N}_{k} - 1} {\sum }_{i = 1}^{{N}_{k}} {( {X}_{ki} - {\overline{X}}_{k} )}^{2}$$

(5) Compute the pooled mean and standard deviation of all SMBG readings as follows:

$$\overline{X} = \frac{1}{M} {\sum }_{k = 1}^{M} {\sum }_{i = 1}^{{N}_{k}} {X}_{ki}$$

$${SD}_{p}^{2} = \frac{( {N}_{1} - 1 ) {SD}_{1}^{2} + ( {N}_{2} - 1 ) {SD}_{2}^{2} + … + ( {N}_{k} - 1 ) {SD}_{k}^{2}}{{N}_{1} + {N}_{2} … {N}_{k} - k}$$

(6) For each time bin k compute a deviation contrast (“tₖ”):

$${t}_{k} = \frac{{X}_{k} - \overline{X}}{\sqrt{\frac{{SD}_{p}^{2}}{N} + \frac{{SD}_{k}^{2}}{{N}_{k}}}}$$

Alternatively, the deviation contrast can be computed using the grand mean via the formula

$${t}_{k} = \sqrt{N} \frac{{X}_{k} - {Y}_{k}}{SD 1}$$

where Yₖ is the average of the means in the 4 time bins other thank and SD1 is an estimate of the SD of Xₖ−Yₖ. For example, for k=₂ Y₂=¼(X+X₃+X₄+X₅)

Given the null hypothesis that the mean in time bin k is not higher than the means in the other time bins, the statistic tₖ will be distributed in a manner that is close to a t-distribution, which for N>30 can be approximated by a central normal distribution. In the validation data set the average absolute error of this approximation was 0.0009 (SD=0.001), thus the normal approximation is acceptable for the practical implementation of the algorithms. (NOTE: computing directly a t-distribution can be difficult, which is a reason for using a normal approximation in certain exemplary embodiments). The normal approximation of the probability that tₖ>0 can be computed as P (tₖ>0)=Φ(tₖ), where Φ(tₖ) is the distribution function of a central normal distribution (with mean zero and SD=1).

(7) Φ(tₖ) can be approximated by a polynomial using the following code with z=tₖ:
static double NormalCDF(double z)

{

if (z > 6) return 1.0;
if (z < −6) return 0.0;
double b1 = 0.31938153;
double b2 = −0.356563782;
double b3 = 1.781477937;
double b4 = −1.821255978;
double b5 = 1.330274429;
double p = .2316419;
double c2 = 0.3989423;
double a = Math.Abs(z);
double t = 1.0 / (1.0 + a * p);
double b = c2 * Math.Exp(−z * z/ 2);
double Φ = ((((b5*t+b4) * t+b3) * t+b2) * t+b1) * t;

Φ = 1.0 − b * CDF;

if (z< 0) Φ = 1.0 − Φ;
return n;

}

Other approximations of the central normal cumulative distribution function (CDF) can be used.

For Hyperglycemia:

Compute the probability (“Pₖ”) of BG exceeding a certain preset threshold α1 (e.g. α1=180 mg/dl) computed as:

$${P}_{k} ( α 1 ) = \frac{1}{{N}_{k}} {\sum }_{i = 1}^{{N}_{k}} {I}_{ki} ( α 1 )$$

$$where$$

$${I}_{ki} ( α 1 ) = \{ \begin{matrix}1 , & if {X}_{ki} > α 1 \\ 0 , & if {X}_{ki} \leq α 1\end{matrix}$$

b. Compute the individual composite probability (“CPₖ”) the average BG in a time bin k to exceed the preset threshold α1 (e.g. α1=180 mg/dl) and the mean BG in time bin k to be higher than the rest of the bins (or than the grand mean): CPₖ(α1)=Pₖ(α1)·Φ(tₖ);
c. If the composite probability CPₖ (α1) exceeds a certain threshold 31 (e.g. β1=0.5), identify the time slot k as a period of increased risk for hyperglycemia.

For Hypoglycemia:
a. Compute the probability of BG to be lower than a certain threshold α2 (e.g. α2=70 mg/dl):

$${P}_{k} ( α 2 ) = \frac{1}{k} {\sum }_{i = 1}^{{N}_{k}} {I}_{ki} ( α 2 )$$

$$where$$

$${I}_{ki} ( α 2 ) = \{ \begin{matrix}1 , & if {X}_{ki} < α 2 \\ 0 , & if {X}_{ki} \geq α 2\end{matrix}$$

b. Compute the individual composite probability the average BG in a time bin k to be lower than the threshold α2 (e.g. α2=70 mg/dl) and the mean BG in time bin k to be lower than the rest of the bins (or than the grand mean):

CPₖ(α2)=Pₖ(α2)(1−Φ(tₖ));
c. If the composite probability CPₖ (α2) exceeds certain threshold β2 (e.g. β2=0.1), identify the time slot k as a period of increased risk for hypoglycemia and issue a message.

Algorithm Identifying Patterns of Increased Glucose Variability

The logic of the algorithm identifying patterns of increased glucose variability is similar to the logic of the algorithm identifying patterns of hyperglycemia. Instead of average BG, however, the test includes a measure of variability in each time bin. For example, such a measure could be the SD of BG, or the risk standard deviation (“RSD”) of these values converted into risk space. In this embodiment, we use the RSD because this measure is equally sensitive to hypoglycemic and hyperglycemic glucose variability.

The overall variability of a person is computed using the ADRR, but can be also computed using the overall standard deviation of SMBG readings, a logarithmic transformation of the deviation of glycemia from an arbitrary assigned “ideal” glucose value (“M-value”), mean amplitude of glycemic excursions (“MAGE”), lability index, or any other accepted measure of variability. The standard deviation of SMBG readings would make the variability profile more sensitive to hyperglycemic excursions and less sensitive to hypoglycemia. In this embodiment, we use the ADRR because this measure of variability has been shown to be superior in terms of its sensitivity to, and predictive ability of extreme glycemic excursions, and because it has clearly identified population thresholds.

As in the previous section, the glucose readings in each time bin are evaluated for deviations towards higher variability. These deviations are assessed for exceeding idiosyncratic threshold determined via analysis of the glycemic patterns of each individual. In addition, the overall ADRR of a person is classified with respect to population parameters. The combination of idiosyncratic deviations and overall ADRR can be used to declare time periods as high risk for increased variability. The judgment of each of the conditions is governed by two parameters:
ADRR threshold parameter (α), reflecting population-level definition of high glucose variability. For example, α=30, or α=40 mg/dl can be acceptable values.
Individual probability threshold (β) reflecting the idiosyncratic chance that variability would be high for this particular individual during this particular period of time. For example, β=0.6 to 0.8 can be acceptable values.

Steps of the Algorithm:

(1) Transform each BG reading into “risk space” using a formula: f (BG, a, b)=c. [(In (BG))^{a}−b}], where the parameters of this function depend on the BG scale and are as follows: if BG is measured in mg/dl, then a=1.084, b=5.381, c=1.509. If BG is measured in mmol/l, then a=1.026, b=1.861 and c=1.794.

(2) For each time bin, compute the risk standard deviation, RSD, using the formula:

$${RSD}_{k}^{2} = \frac{1}{{N}_{k} - 1} {\sum }_{i = 1}^{{N}_{k}} {( f ( {X}_{ki} ) - f {\overline{X}}_{k} )}^{2}$$

$$where$$

$$f {\overline{X}}_{k} = \frac{1}{{N}_{k}} {\sum }_{k = 1}^{{N}_{k}} f {X}_{ki}$$

(3) Compute the overall pooled RSD using the formulas above, including all SMBG readings across all time bins.

(4) For each time bin compute the ratios Zₖ=5*(RSDₖ/RSD−1). As shown in Reference 1, these ratios have approximately central normal distribution and therefore can be used for testing of idiosyncratic deviations in the same way as in the previous section.

(5) For each time bin, compute each individual's probability that Zₖ>0 as P (Zₖ>0)=Φ(Zₖ), where Φ is the distribution function of central normal distribution computed by the polynomial approximation given in the previous section. P (Zₖ>0) is the individual probability that a certain time bin will have higher variability than the others.

(6) Compute the ADRR of each person as an overall marker of variability, the published algorithm from Diabetes Care, Vol. 29, Num. 11, November 2006. In brief the computation of the ADRR is accomplished by the following formulas:
For each SMBG reading compute r(BG)=10.f(BG)², where f(BG) is defined in (1) above;
Compute rl(BG)=r(BG) if f(BG)<0 and 0 otherwise;
Compute rh(BG)=r(BG) if f(BG)>0 and 0 otherwise.
Let x₁¹, x₂¹, . . . Xₙ¹ be a series of n SMBG readings taken on Day 1;
Let x₁^{M}, x₂^{M} . . . Xₙ^{M} be a series of n^{M} SMBG readings taken on Day M.

Where the number of days of observation is between 14 and 42;

LRⁱ=max (rl(x₁ⁱ), rl(x₂ⁱ), . . . , rl(xₙⁱ)) and
HRⁱ=max (rh(x₁ⁱ), rh(x₂ⁱ), . . . , rh(xₙⁱ)) for day #i; i=1,2, . . . M.
The Average Daily Risk Range is then defined as:

$$ADRR = \frac{1}{M} {\sum }_{i = 1}^{M} [ {LR}^{i} + {HR}^{i} ]$$

4. Evaluation and Messaging Procedure

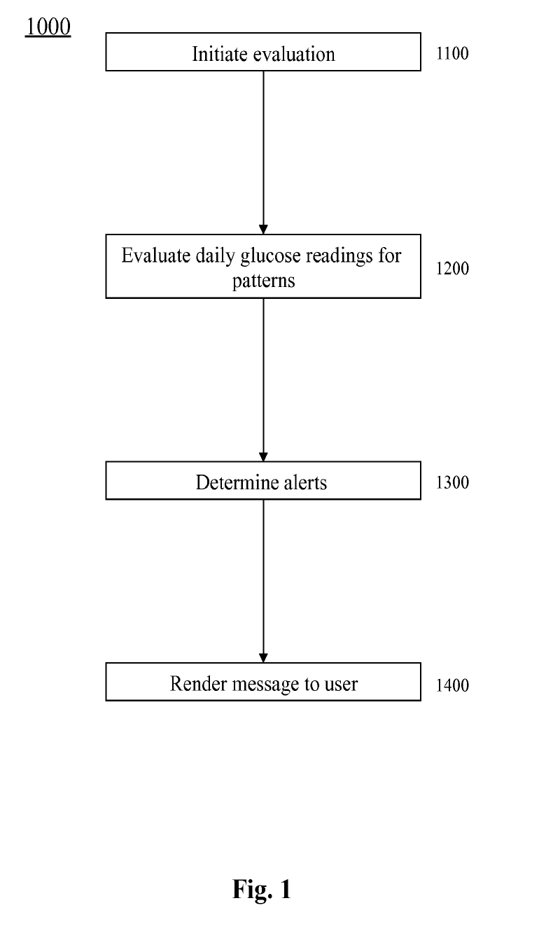

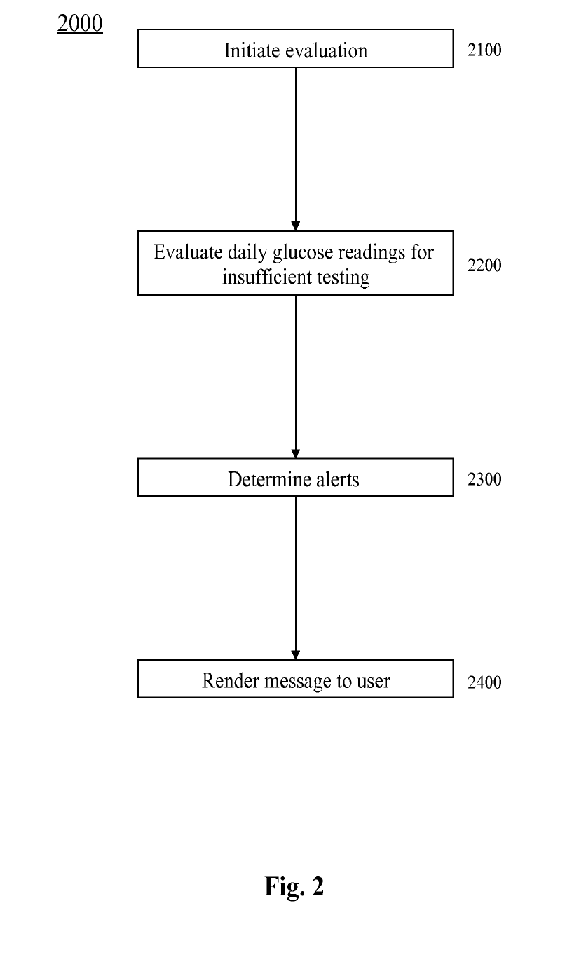

In a preferred embodiment, the evaluation of future time periods occurs when a patient tests their blood glucose levels. At that time, one or more future time periods of the day are evaluated to determine if there are deleterious glucose patterns or inadequate testing patterns. For any deleterious glucose pattern or inadequate testing pattern identified in the time period subsequent to when the evaluation is taking place, the system will be programmed to provide an alert to the user at the beginning of the time period with inadequate testing, or about an hour before the time period with deleterious glucose patterns. The system then provides the user with this alert at the programmed time once that time is reached in that day or the subsequent day. FIG. 1 and FIG. 2 describe this process for deleterious glucose patterns and inadequate testing patterns respectively.

Also in the preferred embodiment, in the event there is an inadequate testing pattern in a second time period subsequent to the time period wherein the evaluation is taking place, a third time period subsequent to the second time period is evaluated for deleterious glucose patterns, and if it is determined that deleterious glucose patterns exist, the user will be notified in the first time period of inadequate testing in the second time period as well as deleterious glucose patterns in the third time period. The impetus for this has been supported by the calculations outlined in Tables 2 and 3, which show a greater propensity for deleterious glucose patterns occurring after a period of inadequate testing. In addition, if this third time period also is a period of inadequate testing, a fourth time period of the day, subsequent to the third time period, will be evaluated for deleterious glucose patterns, and if it is determined that deleterious glucose patterns exist, the user will be notified in the first time period of inadequate testing in the second and third time periods followed by a period of deleterious glucose patterns.

The method of the invention may be implemented using hardware, such as hardware specific processors and integrated circuits, and software such as computer programs and computer program logic, and devices with communication infrastructure and blood glucose measurement systems or a combination thereof and may be implemented in one or more computer systems or other processing systems, such as a mobile device, watch, handheld computer, tablet computer, desktop computer, laptop computer, insulin pump controller, key fob, body-wearable technology accessory, smartphone, and/or a blood glucose monitoring device equipped with adequate processing capabilities and memory, etc.

FIG. 1 is a flowchart of an exemplary embodiment of a method adapted to evaluate daily time periods for deleterious patterns of glycemic control, saving the identified time periods of deleterious glycemic control, and then later initiating a program for sending a message to a user about the pattern of glycemic control more proximal to period in which the pattern of glycemic control typically manifests itself. In certain exemplary embodiments, a system can be programmed to provide a message or alert in the future about when to test based on analysis of daily time periods wherein deleterious glucose control has been identified. At activity 1100, as a result of a blood glucose reading, a pre-programmed alert, or a user prompt, in a first period of time (e.g., morning) a system can initiate an evaluation of subsequent time periods of the day. At activity 1200, each daily time period over 24 hours, or at least over the next 8-24 hours, can be evaluated to determine time periods where deleterious glucose patterns exist. For example, daily glucose readings can be evaluated for deleterious glucose patterns in subsequent daily time periods (e.g., lunch, afternoon, evening, and/or night). Frequent hyperglycemia can be determined relative to an absolute threshold and/or relative to rest of day. Frequent hypoglycemia can be determined relative to absolute threshold and/or relative to rest of day. Frequent variability can be determined relative to absolute threshold and/or relative to rest of day. At activity 1300, the system can program an alert to instruct the user to test at the beginning of, or immediately prior to, the deleterious glucose pattern time period to aid the user in determining the cause of the deleterious glucose pattern. The system can program alerts for any time period where deleterious glucose patterns have been identified. Activities 1100, 1200, and 1300 can take place in the first period of time. At activity 1400, before or during the future time period in which the pattern is identified, the pattern can be identified and the user alerted to test at about that time period. The system can message the user about any deleterious glucose patterns before or during the time period for which the deleterious glucose pattern has been identified. The alert to test could be up to four hours before the time period with the deleterious glucose pattern, or up to two hours into the period with the deleterious glucose pattern. The alert would preferably be given between two hours before to the beginning of the deleterious glucose pattern time period. A prompt for a specific desired test time can be given to the user at the time of the evaluation of the subsequent daily time periods, or at the user's discretion, and alerts for testing can also be turned off by the user in the event the user does not want to be notified.

FIG. 2 is a flowchart of an exemplary embodiment of a method adapted to evaluate daily time periods for periods of inadequate testing, saving the identified time periods of inadequate testing, and then later initiating a program for sending a message to a user about the period of inadequate testing more proximal to period of inadequate testing. The system can be programmed to provide a message or alert about when to test based on analysis of daily time periods of inadequate glucose testing in the future. At activity 2100, as a result of a blood glucose reading, a pre-programmed alert, or a user prompt, in a first period of time (e.g., morning) the system initiates an evaluation of one or more subsequent time periods of the day. At activity 2200, each subsequent daily time period over the next 8-24 hours can be evaluated to determine time periods where inadequate testing exist. Daily glucose readings can be evaluated for inadequate glucose testing in subsequent daily time periods (e.g., lunch, afternoon, evening, and night). A determination can be made that a count of readings is below a predetermined threshold. A determination can be made that % of total number of readings is below a predetermined threshold. A determination can be made that a count and/or a percentage of days in recent past with readings is below a predetermined threshold. At activity 2300, the system can program an alert to test at the beginning of or immediately prior to, the period of inadequate testing to aid the user in obtaining beneficial glucose testing information in periods where there is a deficiency. The system can program alerts for any time period where inadequate glucose testing patterns have been identified. Activities 2100, 2200, and 2300 can take place in the first period of time. At activity 2400, before or during the future time period in which the pattern is identified, the pattern can be messaged to the user and the user is alerted to test at about that time period. The system can message the user about any inadequate glucose testing patterns before or during the time period for which the inadequate glucose testing pattern has been identified. The alert about the identified pattern could be up to two hours before the time period with the inadequate glucose testing or up to four hours into the period with the inadequate glucose testing. The alert would preferably be given within an hour of the beginning of the period of inadequate glucose testing. A prompt for a specific desired test time can be given to the user at the time of the evaluation of the subsequent daily time periods, or at the user's discretion, and alerts for testing can also be turned off by the user in the event the user does not want to be notified.

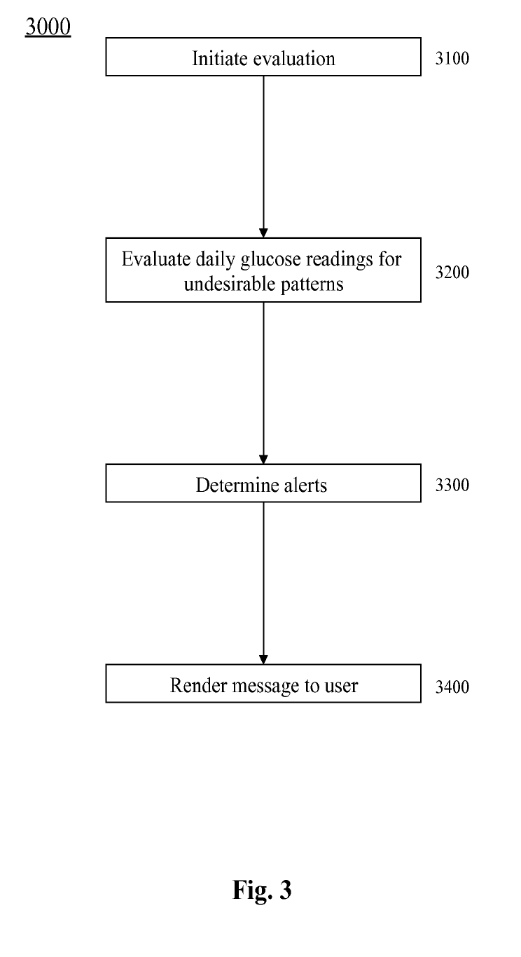

FIG. 3 is a flowchart of an exemplary embodiment of a method adapted to, in a first period of time, evaluate a second period of time to determine if there is inadequate testing, and if there is inadequate testing, evaluating a third period of time for patterns of deleterious glucose control, and then displaying a message to a user in the first period of time about the inadequate testing in the second period of time and any deleterious glucose control in the third period of time. At activity 3100, in a first time period (e.g., in the morning), the system can initiate an evaluation of future time periods. At activity 3200, a subsequent second time period of the day can be evaluated to determine if there are undesirable testing frequency patterns in that second time period. If the system determines there are undesirable testing frequency patterns in the second time period, the system will evaluate a subsequent third time period of the day to determine if there are deleterious glucose patterns in the third time period of the day. If there are deleterious glucose patterns in the third time period of the day, the system can inform the user in the first time period of the day of the undesirable testing frequency patterns in the second time period and deleterious glucose patterns in the third time period of the day. The system can evaluate daily glucose readings for undesirable testing patterns in the second time period (e.g., lunch). A determination can be made whether a count of readings is below a predetermined threshold, whether a percentage of a total number of readings is below a predetermined threshold, whether a count or percentage of days in the recent past with readings is below a predetermined threshold. The system can evaluate daily glucose readings for deleterious glucose patterns in the third time period (e.g., afternoon). The system can evaluate frequent hyperglycemia relative to an absolute threshold and/or relative to the rest of the day. The system can evaluate frequent hypoglycemia relative to an absolute threshold and/or relative to the rest of the day. The system can evaluate frequent blood glucose variability relative to an absolute threshold and/or relative to the rest of the day. This allows the user to know of suboptimal testing patterns before they tend to materialize. Furthermore, they will be informed that these suboptimal testing patterns are resulting in deleterious glucose control in the period following the period of deficient testing. At activity 3300, alerts can be determined for time periods subsequent to the first time period. At activity 3400, before or during a future time period in which a pattern is identified, the pattern can be messaged to the user and the user is alerted to test at about that time period. The system can display a message about undesirable testing patterns. For example, the message can comprise information about undesirable testing patterns in the second time period and/or deleterious glucose patterns in the third time period.

FIG. 4 is a flowchart of an exemplary embodiment of a method. At activity 4100, in a first period of time, the system can initiate an evaluation. At activity 4200, the system can evaluate a second period of time to determine if there is inadequate testing, and if there is inadequate testing, evaluating a third period of time to determine if there is inadequate testing, and if there is inadequate testing, evaluate a fourth period of time for patterns of deleterious glucose control, and then displaying a message to a user in the first period of time about the inadequate testing in the second and third periods of time and any deleterious glucose control in the fourth period of time. The system can evaluate daily glucose readings for undesirable testing patterns in the second time period. The system can determine if a count of readings is below a predetermined threshold, if a percentage of a total count of readings is below a predetermined threshold, and/or if a count or percentage of days in the recent past with readings is below a predetermined threshold. The system can evaluate daily glucose readings for undesirable testing patterns in the third time period. The system can determine if a count of readings is below a predetermined threshold, if a percentage of a total count of readings is below a predetermined threshold, and/or if a count or percentage of days in the recent past with readings is below a predetermined threshold. At activity 4300, the system can evaluate daily glucose readings for deleterious glucose patterns in the third time period. The system can determine frequent hyperglycemia relative to absolute threshold and/or relative to the rest of the day, frequent hypoglycemia relative to absolute threshold and/or relative to the rest of the day, and/or frequent blood glucose variability relative to absolute threshold and/or relative to the rest of the day. After an exemplary system has evaluated the second time period for deleterious glucose patterns, the system can evaluate the third time period to determine if there are deleterious glucose patterns in that third time period, and if there are the system can evaluate a subsequent fourth time period of the day to determine if there are deleterious glucose patterns in that fourth time period, and then if there are inform the user in the first time period about deleterious glucose patterns in the second and third time periods followed by a period of deleterious glucose patterns in the fourth time period of the day. This allows the user to be aware of a significant problem in their glucose patterns over two time periods of their day in time periods subsequent to the significant deficiency in testing. At activity 4400, alerts can be determined for time periods subsequent to the first time period. At activity 4500, before or during a future time period in which a pattern is identified, the pattern can be messaged to the user. The system can render message about undesirable testing patterns and/or deleterious glucose patterns in the second, third, and/or fourth time periods.

Certain exemplary embodiments provide a method, which can comprise, via a user interface, rendering a message in a first time period of a day that comprises information about at least:
one pattern of insufficient blood glucose testing in a second time period of the day, wherein the second time period subsequent to the first time period; and/or
at least one pattern of deleterious blood glucose in a third time period of the day, wherein the third time period subsequent to the second time period of the day.

The message can be based upon a determination that identifies daily periods with at least one of:
deleterious glucose patterns, the deleterious glucose patterns comprising at least one of an indication of hyperglycemia, hypoglycemia, and glucose variability; and/or
insufficient blood glucose testing during one or more time periods out of a plurality of time periods during the day.

The determination of insufficient blood glucose testing can comprise at least one of:
whether a percent of total readings in a first predetermined time period is below a first predetermined threshold; and/or
whether a count of readings in a predetermined count of days is below a second predetermined threshold.

Insufficient blood glucose testing can also be identified if a percent of total readings in a second predetermined time period is below a third predetermined threshold, wherein the second predetermined time period is shorter than the first predetermined time period. The first predetermined time period can be between approximately 14 days and approximately 60 days, and the second predetermined time period can be between approximately 5 and approximately 30 days. The message can be accompanied by at least one instruction or recommendation for a diabetic patient to test in a time period subsequent to the first time period. The determination of daily periods can be initiated by an entry of a blood glucose reading via the user interface.

Certain exemplary embodiments provide a method, which can comprise, in a first time period making a determination of at least one of:
daily periods with deleterious glucose patterns, wherein the deleterious glucose patterns comprising at least one of an indication of hyperglycemia, hypoglycemia, and glucose variability; and/or
insufficient blood glucose testing during one or more time periods out of a plurality of time periods during a day.

The method can comprise saving an indication concerning at least one time period of insufficient blood glucose testing or deleterious glucose patterns adapted for rendering to a user in a second time period. The second time period can be subsequent to the first time period. Via a user interface, a message can be rendered in the second time period of the day about the deleterious glucose patterns or insufficient blood glucose testing patterns occurring in one or more time periods of the day subsequent to the second time period. The message in the second time period of the day can relate to at least one pattern of:
insufficient blood glucose testing in a third time period of the day, wherein the third time period subsequent to the second time period; and/or
at least one pattern of deleterious blood glucose in a fourth time period of the day, wherein the fourth time period subsequent to the third time period of the day.

The determination of insufficient blood glucose testing can comprise at least one of:
determining whether a percent of total readings in a first predetermined time period is below a first predetermined threshold; and/or
determining whether a count of readings in a predetermined count of days is below a second predetermined threshold.

Insufficient blood glucose testing can also be identified if a percent of total readings in a second predetermined time period is below a third predetermined threshold. The second predetermined time period can be shorter than the first predetermined time period. The first predetermined time period can be between approximately 14 days and approximately 60 days, and the second predetermined time period can be between approximately 5 and approximately 30 days. The message can be accompanied by at least one instruction or recommendation for a diabetic patient to test in a time period subsequent to the second time period. The determination of daily periods can be initiated by an entry or acceptance of a blood glucose reading via the user interface.

Certain exemplary embodiments provide a machine-readable medium comprising machine-implementable instructions for activities. The activities can comprise via a user interface, rendering a message in a first time period of a day about insufficient blood glucose testing in a second time period of the day and about at least one pattern of deleterious blood glucose in a third time period of the day. The second time period can be subsequent to the first time period. The third time period of the day can be subsequent to the second time period of the day. The message can be based upon an evaluation of blood glucose readings stored in a memory device that determines daily periods with at least one of:
deleterious glucose patterns, the deleterious glucose patterns comprising at least one of an indication of hyperglycemia, hypoglycemia, and glucose variability; and/or
insufficient blood glucose testing during one or more time periods out of a plurality of time periods during the day.

The message can comprise information concerning:
at least one pattern of insufficient blood glucose testing in a time period of the day subsequent to the second time period; and/or
at least one pattern of deleterious blood glucose in the third time period of the day that is subsequent to the second time period of the day.

The message can be rendered responsive to a determination of at least one of:
a percentage of total readings in a first predetermined time period is below a first predetermined threshold; and/or
a count of readings in a predetermined count of days is below a second predetermined threshold.

The message can be rendered responsive to a determination that a percentage of total readings in a second predetermined time period is below a third predetermined threshold and/or a determination that the second predetermined time period is shorter than the first predetermined time period. The message can be rendered responsive to a determination that the first predetermined time period is between approximately 14 days and approximately 60 days, and the second predetermined time period is between approximately 5 and approximately 30 days. The activities can comprise rendering at least one instruction or recommendation, via the user interface, for a diabetic patient to test in a time period subsequent to the first time period. The activities can comprise receiving a blood glucose reading through the user interface to initiate the determination of daily periods.

Certain exemplary embodiments provide a method, which can comprise via a user interface, rendering a message in a first time period of a day about at least one pattern of:
insufficient blood glucose testing; and/or
deleterious blood glucose, wherein the deleterious glucose patterns can comprise at least one of an indication of hyperglycemia, hypoglycemia, and glucose variability.

The message can communicate, substantially in one display, for every time period of the day, an identification of any patterns of insufficient blood glucose testing or deleterious blood glucose. The message can be based upon a determination that identifies the at least one pattern during one or more time periods out of a plurality of time periods during the day.

Certain exemplary embodiments provide a method, which can comprise via a user interface, rendering a message in a first time period of a day that comprises information about at least one insufficient blood glucose testing pattern in one or more time periods of the day subsequent to the first time period. The message can be based upon a determination that identifies at least one of:
daily periods with deleterious glucose patterns, wherein the deleterious glucose patterns can comprise at least one of an indication of hyperglycemia, hypoglycemia, and glucose variability; and/or
insufficient blood glucose testing during one or more time periods out of a plurality of time periods during the day.

The insufficient blood glucose testing can be determined by criteria comprising whether a count of readings in a predetermined count of days is below a predetermined threshold.

Certain exemplary embodiments provide a method, which can comprise via a user interface, rendering a message in a first time period of a day that comprises information about at least one insufficient blood glucose testing pattern in one or more time periods of the day subsequent to the first time period. The message can be based upon a determination that identifies daily periods with at least one of:
deleterious glucose patterns, the deleterious glucose patterns comprising at least one of an indication of hyperglycemia, hypoglycemia, and glucose variability; and/or
insufficient blood glucose testing during one or more time periods out of a plurality of time periods during the day.

The insufficient blood glucose testing can be determined by whether a percent of total readings in a first predetermined time period is below a first predetermined threshold, or whether a percent of total readings in a second predetermined time period is below a second predetermined threshold. The second predetermined time period can be shorter than the first predetermined time period.

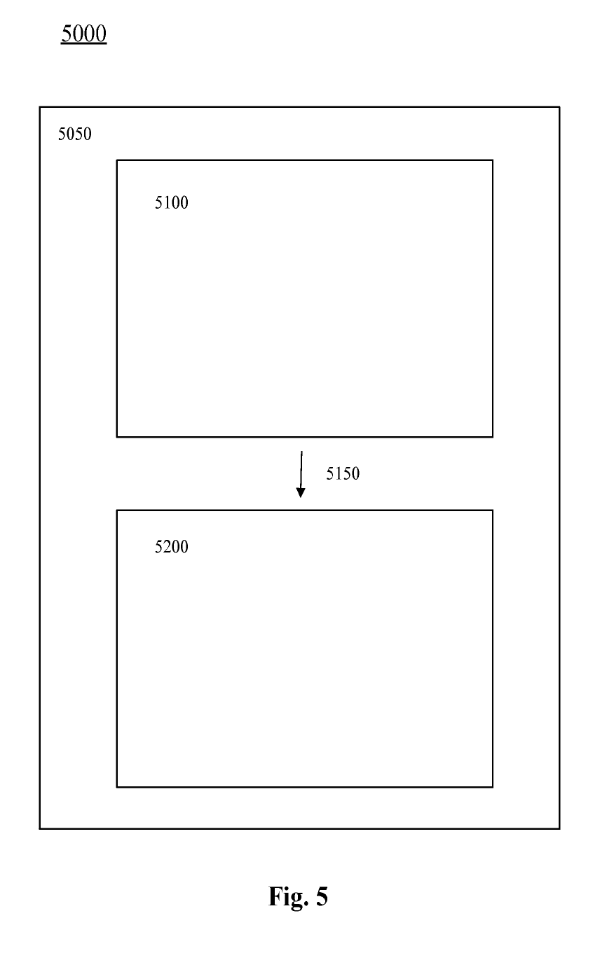

FIG. 5 is a display of an exemplary output of an initial message 5100 and more detailed information 5200 relating to inadequate testing in a second period of time followed by a deleterious glucose pattern in a third period of time. Initial message 5100 can be conveyed to the user, and can comprise an alert notifying the user of a period of inadequate testing followed by a period of deleterious glucose patterns, and can be communicated to the user via a user interface 5050 and can provide information to the user about patterns and timing, via text, graphical, audible means or vibrations means, but preferably via text and graphical means. An exemplary initial message might state “You have a period of infrequent testing at lunch, following by a pattern of high sugar levels in the afternoon.” In certain exemplary embodiments, the user can access more detailed information 5200 about the identified patterns on a separate screen or interface via interaction with a user communication interface such as a touch screen 5150. An exemplary more detailed information rendering might state “You have only tested 3 times between 11:00 am and 3:00 pm in the last 30 days (only 2% of all testing occurs between 11:00 am and 3:00 pm in the last 30 days). In the last 30 days your average blood sugar is 215 mg/dL between 3:00 an 7:00 pm.”

In certain exemplary embodiments, the daily time periods can be pre-defined by the user or by the user's device or data management system, and can take the form of 2-12 hour time blocks most conducive to periods of the day consistent with patient behavioral patterns. The preferred time periods consist of four hour time periods and an eight hour time period for night time (sleeping). This allows for sufficient results for evaluation to be collected in each time period over multiple days and also matches with typical idiosyncratic patterns of behavior in the nighttime, morning, at mid day, in the afternoon and in the evening.

In certain exemplary embodiments, the undesirable testing frequency patterns can take the form of being below a pre-defined absolute minimum number of glucose readings in a given daily time period, or being below a pre-defined percentage of glucose readings out of the total number of glucose readings, or having a pre-specified number or percentage of glucose readings in that time period over a pre-defined number of days proximal to the day wherein the period is being evaluated. These pre-defined thresholds can be preset in a device and data management system, and are defined by population and sub-population level analyses for people who test at different frequencies, have different levels of glycemic control, or have Type 1 versus Type 2 diabetes.

In certain exemplary embodiments, deleterious glucose patterns can take the form of patterns of hyperglycemia, patterns of hypoglycemia, or patterns of high glucose variability, or some combination thereof. Patterns can take the form of exceeding a predefined number of days with a hypoglycemic or hyperglycemic episodes in that time period, or calculating probabilities of a pattern forming relative to an absolute clinical threshold for hyperglycemia, hypoglycemia or variability, or calculating probabilities relative to a probability threshold for the entire day, or preferably a combination of both. Variability can be measured by common statistical tools such as standard deviation, variance, range, or variability measures more specific to diabetes such as interquartile range, average daily risk range, mean amplitude of glycemic excursions, M value, or other, but preferably the average daily risk range.

In certain exemplary embodiments, to accommodate idiosyncratic behaviors and personalized testing regimens the user can change a number of configurable settings around the desired messages received. The user can decide to not receive messages about undesirable testing patterns at all, or only in certain periods of the day. In addition, the user can change the individualized thresholds for percentages of readings desired in a given time period, or absolute number of readings desired in a given time period, or a minimum number of days with testing data in that time period evaluable.

In certain exemplary embodiments, the evaluation of testing patterns, deleterious glucose control periods and receipt of feedback can be initiated when the user tests their blood sugar levels, via a button press, or via some other initiating input from the user. This allows for patients to obtain feedback when they typically interact with a diabetes management device such as a blood glucose monitor or data management system. Thus when they are engaged in evaluating their most current glucose result in the context of a treatment decision such as how much insulin to dose, how much to eat (and what to eat), and how much or how little to exercise to keep blood glucose levels within an optimal range. Alternatively, the evaluation of testing patterns and periods of deleterious glucose control can be initiated by an alert set by the system manufacturer or the user.

In another aspect of the present invention, a user can receive feedback on testing patterns and deleterious glucose control patterns on their blood glucose monitoring system or a data management system. A data management system in accordance with the present invention can include a personal digital assistant (“PDA”), phone, blood glucose monitoring device, tablet, watch, insulin pump, insulin doser, internet interface, optical viewing device, or other media. The evaluation of patterns can also be performed remotely from the user's device and data management system, and then the feedback given at a time when the user's has access to their device or data management system.

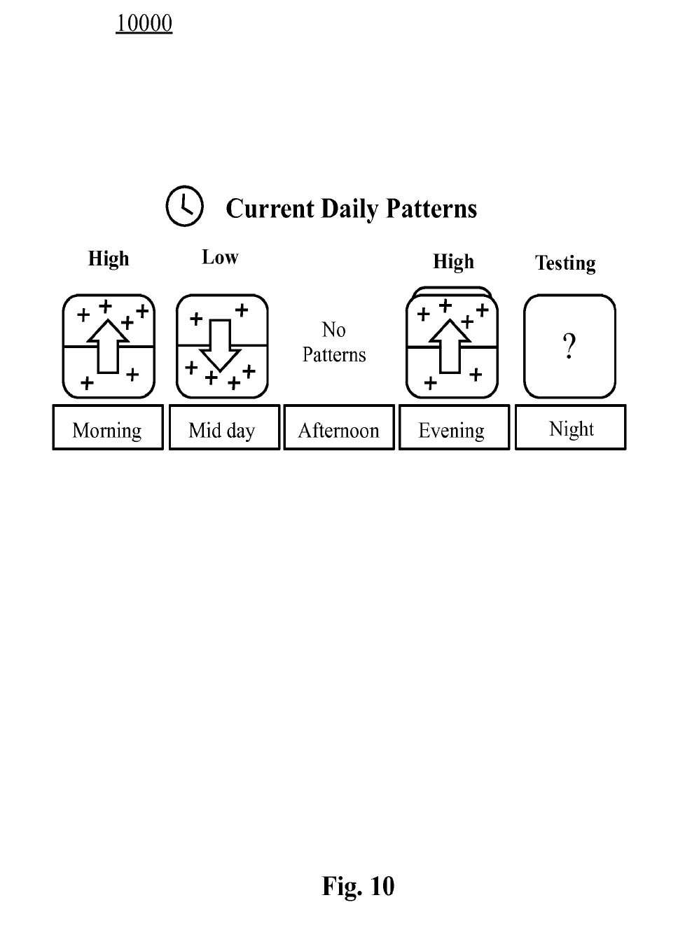

In certain exemplary embodiments, as shown in FIG. 10, a presentation of all current daily deleterious patterns and periods of inadequate testing by time period can be provided to the user, so that the user can initiate an evaluation to understand where they have deleterious patterns and where they have testing deficiencies over the course of the entire day at any given period of time.

In certain exemplary embodiments, a user can evaluate blood glucose readings organized chronologically or reverse chronologically within daily time periods, so that the underlying cause of patterns within a specific time period, and the blood glucose readings leading to the generation of the patterns, can be isolated and scrutinized.

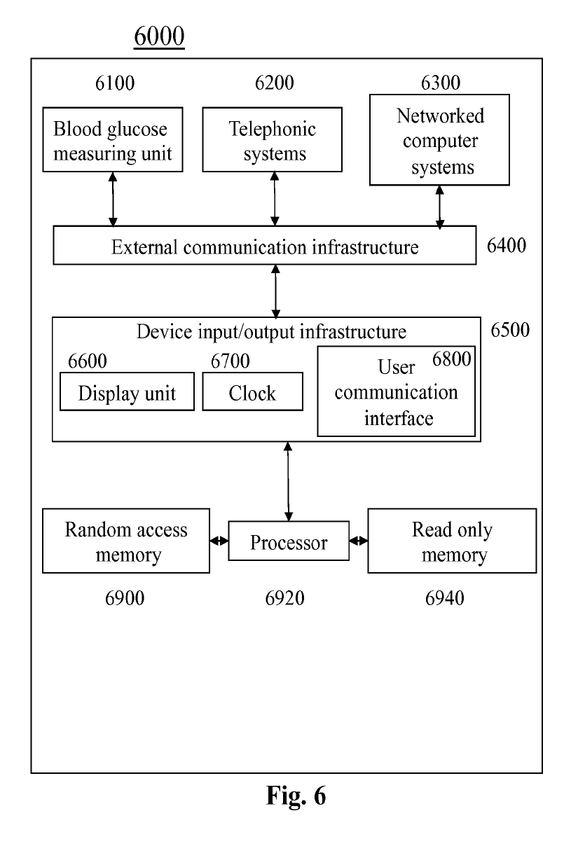

FIG. 6 is a diagram of exemplary hardware and functional components of a system that can deliver information to the user about periods of inadequate testing and related patterns of deleterious glycemic control.

As illustrated in FIG. 6, certain exemplary embodiments may be implemented in a data management system 6000 that interfaces with a blood glucose measurement unit 6100 via a unit directly embedded in the data management system or via wire line ports, infrared, fiber optic cable, RF, Bluetooth, RS 232, and/or USB cables. An external communication interface 6400 can accept blood glucose measurement data from the blood glucose measuring unit 6100, and may also have protocols for interfacing with telephonic systems 6200 such as a cellular or hardwire modem, telephone, or fax, and also with networked computer systems 6300 such as a local area network, or the Internet also via wire line ports, infrared connections, fiber optic cables, RF, Bluetooth, and/or USB cables. The external communication interface 6400 can be adapted to convert information and signals, digital or analog, from external devices and systems into a local machine readable format in the device input/output infrastructure 6500, and vice versa.

The device input/output infrastructure 6500 can also pre-prepare local machine readable information via encryption or other methods into pre-specified formats for certain users of external systems such as blood glucose measuring units 6100, telephonic systems 6200 and networked computer systems 6300 and also so that it can easily be modified and transmitted by the external communication interface 6400 or accept encrypted or modified data that it can decipher for processing or saving in the read only memory or random access memory. In addition, device input/output infrastructure 6500 preferably has a display unit 6600 such as a monitor, speaker, screen, or projector for the user to receive information from the system and preferably a user communication interface 6800 such as a keyboard, mouse, touchpad, buttons, microphone, or camera that allows human control and command of the data management system.

A digital or analog clock 6700 with up to date year, day, hour, minute and second information may be accessible through external communication infrastructure 6400, or embedded in other system components, but is preferably located in the device input/output infrastructure 6500, and preferably is remotely adjustable via communication through the external communication interface 6400, display unit 6600, or user communication interface 6800.

One or more processors 6920 can access instructed program information embedded in the random access memory 6900 and read only memory 6940 such as a cartridge, memory chip, hard disk drive, flash memory drive, floppy disk drive, magnetic tape drive or optical disk drive, to run programs and route information as instructed, to, from and within device input/output infrastructure 6500, as well as access information from clock 6700. Processor 6920 can process information for output via display unit 6600, through user communication interface 6800 or through external communication infrastructure 6400.

In an exemplary system, the data management system collects the glucose data from the glucose measurement unit 6100, stores it on a computer readable medium such as a read only memory 6940 or random access memory 6900 at the time indicated on clock 6700, uses processor 6920 to run a computer program on a computer readable medium that evaluates the newly acquired and stored glucose data, and then if a message is issued according to the program, outputs the message via either the display unit 6600, user communications interface 6800, or the external communications infrastructure 6400.

In an alternative embodiment, at a pre-specified time on clock 6700 a program is run. Processor 6920 evaluates stored glucose data on read only memory 6940 and determines if a message should be provided to the user on display unit 6600 or via external communications interface 6400. If the program determines a message should be displayed, the message is displayed at the current time, or if appropriate at a later time contemporaneous with a period of time with inadequate testing or deleterious testing patterns. When the message is received by the user, the user can manipulate user communications interface 6800 to learn further details about the message, and obtain contextual information concerning the patterns identified via the display unit 6600.

In another alternative embodiment, the blood glucose data obtained from a blood glucose measuring unit 6100 is saved in a read only memory 6940 and processed by processor 6920 located external to the device input/output infrastructure 6500, on an external telephonic system 6200, blood glucose measuring unit 6100, or networked computer systems 6300. In such systems when a blood glucose reading is obtained from a blood glucose measuring unit 6100, or when the evaluation is otherwise initiated by the system, the blood glucose data or an initiation notification is sent to the external device or system and the evaluation is performed using processor 6920, random access memory 6900, and read only memory 6940 that is beyond the external communication infrastructure 6400. Then information is received through the external communication infrastructure 6400 from the blood glucose measuring unit, telephonic systems 6200 or networked computer systems 6300 performing the evaluation, into device input/output infrastructure 6500 about what information should be messaged to the user, and at which time, and device input/output infrastructure 6500 accesses another internal processor 6920 to run a program to proceed to render the message to the user on display unit 6600 at the appropriate time as well as save the information in another read only memory 6940 internal to the device.

The blood glucose measuring unit 6100 can be any device that can function as a blood glucose data acquisition mechanism. The blood glucose measuring or data acquisition mechanism, device, tool or system includes various methods directed towards drawing a blood sample (e.g. via finger prick followed by application to test strip, vacuum, indwelling cannula) for each test, and a determination of the glucose level using an instrument that reads glucose concentrations by photometric, electrochemical, or infrared methods. Various proxies for measuring blood glucose can be used such as measuring interstitial fluid glucose or intradermal glucose.

In another alternative embodiment, the device input/output infrastructure 6500 accesses internal processor 6920 to run a program to proceed to display all current daily patterns to the user, including deleterious glucose patterns and testing deficiency patterns, on display unit 6600 at the appropriate time, and the information is saved in another read only memory 6940 internal to the device.

Certain exemplary embodiments provide a system (e.g., system 6000), which can comprise a means (e.g., processor 6920) for making a determination in a first time period of a day of at least one of:
daily periods with deleterious glucose patterns, the deleterious glucose patterns comprising at least one of an indication of hyperglycemia, hypoglycemia, and glucose variability; and/or
insufficient blood glucose testing by evaluating blood glucose readings stored in a memory device during one or more time periods out of a plurality of time periods during the day.

The system (e.g., system 6000) can comprise a means for storing on a memory device (e.g., random access memory 6900 and/or read only memory 6940) an indication concerning the determination such that information concerning the determination. The information can be renderable in a second time period that is subsequent to the first time period. The system can comprise a means for, via a user interface (e.g., display unit 6600), rendering a message in the second time period about the determination in one or more time periods of the day subsequent to the first time period.

The system (e.g., system 6000) can comprise a means for rendering the message, via the user interface (e.g., display unit 6600), in a second time period of the day when it relates at least one pattern of insufficient blood glucose testing in a subsequent third time period of the day, and at least one pattern of deleterious blood glucose in a fourth time period of the day that is subsequent to the third time period of the day. The system (e.g., system 6000) can comprise a means for rendering the message, via the user interface (e.g., display unit 6600), responsive to a determination of at least one of:
a percent of total readings in a first predetermined time period is below a first predetermined threshold; and/or
a count of readings in a predetermined count of days is below a second predetermined threshold.

The system (e.g., system 6000) can comprise a means for rendering the message, via the user interface (e.g., display unit 6600), if a percent of total readings in a second predetermined time period is below a third predetermined threshold. The second predetermined time period can be shorter than the first predetermined time period. The system (e.g., system 6000) can comprise a means for rendering the message, via the user interface (e.g., display unit 6600), if the first predetermined time period is between approximately 14 days and approximately 60 days, and/or the second predetermined time period is between approximately 5 and approximately 30 days. The system (e.g., system 6000) can comprise a means for rendering the message, via the user interface (e.g., display unit 6600), for a diabetic patient to test in a time period subsequent to the second time period. The system (e.g., system 6000) can comprise a means for entering or accepting a blood glucose reading through the user interface to initiate the determination.

The system (e.g., system 6000) can comprise a means for rendering a message, via a user interface (e.g., display unit 6600), in a first time period of a day about at least one pattern of:
insufficient blood glucose testing; and/or
deleterious blood glucose, the deleterious glucose patterns comprising at least one of an indication of hyperglycemia, hypoglycemia, and/or glucose variability.

The message can communicate, substantially in one display, for every time period of the day, an identification of any patterns of insufficient blood glucose testing or deleterious blood glucose. The message can be based upon a determination of the at least one pattern during one or more time periods out of a plurality of time periods during the day.

Certain exemplary embodiments provide a system (e.g., system 6000), which can comprise a means for rendering a message, via a user interface (e.g., display unit 6600), in a first time period of a day that comprises information about insufficient blood glucose testing pattern in one or more time periods of the day subsequent to the first time period. The message can be based upon a determination that identifies daily periods with at least one of:
deleterious glucose patterns, wherein the deleterious glucose patterns comprising at least one of an indication of hyperglycemia, hypoglycemia, and glucose variability; and/or
insufficient blood glucose testing patterns during one or more time periods out of a plurality of time periods during the day.

The insufficient blood glucose testing can be determined based upon criteria comprising whether a count of readings in a predetermined count of days is below a first predetermined threshold.

Certain exemplary embodiments provide a system (e.g., system 6000), which can comprise a means for rendering a message, via a user interface (e.g., display unit 6600), in a first time period of a day that comprises information about insufficient blood glucose testing pattern in one or more time periods of the day subsequent to the first time period. The message can be based upon a determination that identifies daily periods with at least one of:
deleterious glucose patterns, the deleterious glucose patterns comprising at least one of an indication of hyperglycemia, hypoglycemia, and/or glucose variability; and
insufficient blood glucose testing patterns during one or more time periods out of a plurality of time periods during the day.

The insufficient blood glucose testing can be determined by whether a percent of total readings in a first predetermined time period is below a first predetermined threshold, or whether a percent of total readings in a second predetermined time period is below a second predetermined threshold. The second predetermined time period can be shorter than the first predetermined time period.

Certain exemplary embodiments message people about testing patterns, identifying where there may not be sufficient information to generate a hyperglycemia or hypoglycemia pattern. Certain exemplary embodiments evaluate and send messages about prospective time periods, so that the user has the opportunity to take action and remedy any undesirable pattern of testing or glucose levels. Certain exemplary embodiments combine the prospective test frequency evaluation with an indication of its possible impact on deleterious glucose patterns in a subsequent period.

Reference 1 discloses a system that provides real-time messages to patients with diabetes about blood glucose patterns. It describes a system that provides messages about deleterious glucose patterns or insufficient testing in a subsequent period of time when a patient tests. Any patterns of insufficient testing are identified via an evaluation of a percentage of overall tests. The methods disclosed in this patent application are novel relative to Reference 1 in that 1) different methods of messaging are proposed such as identifying deleterious patterns that occur after one or more periods of suboptimal testing, and programming alerts to test at or near time periods of suboptimal testing, 2) novel methods of saving and displaying test deficiency and deleterious glucose pattern information are presented, and 3) the methods for identifying suboptimal testing patterns, such as by evaluating more testing in more proximal time periods, are superior in so far as they are more likely to identify test deficiencies that precede periods of deleterious glucose control as shown in Table 2.

The impetus for the reduction to practice of these novel methods over Reference 1 were discovered through new data analysis in 436 patients with diabetes disclosed in Tables 2 and 3, and thus determined to be an important messaging paradigm, undisclosed in Reference 1. As shown in Tables 2 and 3, periods of deleterious glucose patterns succeeding periods of inadequate glucose testing are of greater prevalence than in periods that to not follow periods of inadequate glucose testing. Thus these prevalent patterns might be missed by the methods disclosed in Reference 1 because there could be insufficient data in the period prior to this deleterious glucose pattern to generate a message about the period in which the deleterious glucose pattern presents itself. In addition, methods for evaluating periods of insufficient testing in Reference 1 can be less effective than the novel methods disclosed here, as the novel methods identify a broader range of time periods with suboptimal testing, and which are more likely to precede periods of deleterious glycemic control as shown in Table 3. Furthermore, in the methods disclosed in the Reference 1, for those patients that test infrequently, if several time periods had inadequate data they would not receive feedback about when to test because they would not be testing in those time periods, whereas with the novel methods the evaluation of subsequent time periods can be pre-programmed by the system or user so that alerts can be set to test at appropriate times, regardless of when the user would or would not test.

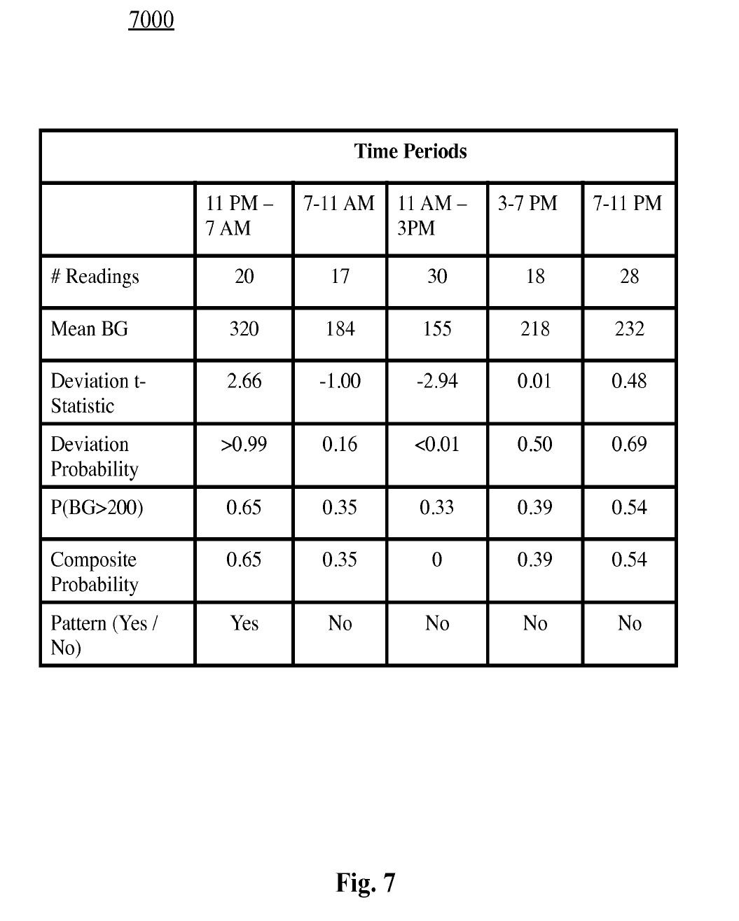

FIG. 7 is a table of exemplary statistical values identifying temporal patterns of hyperglycemia, illustrated by the identification of the overnight period as a period with a pattern of hyperglycemia when the BG threshold parameter is set at 200 mg/dL and the composite probability threshold is set at 0.6.

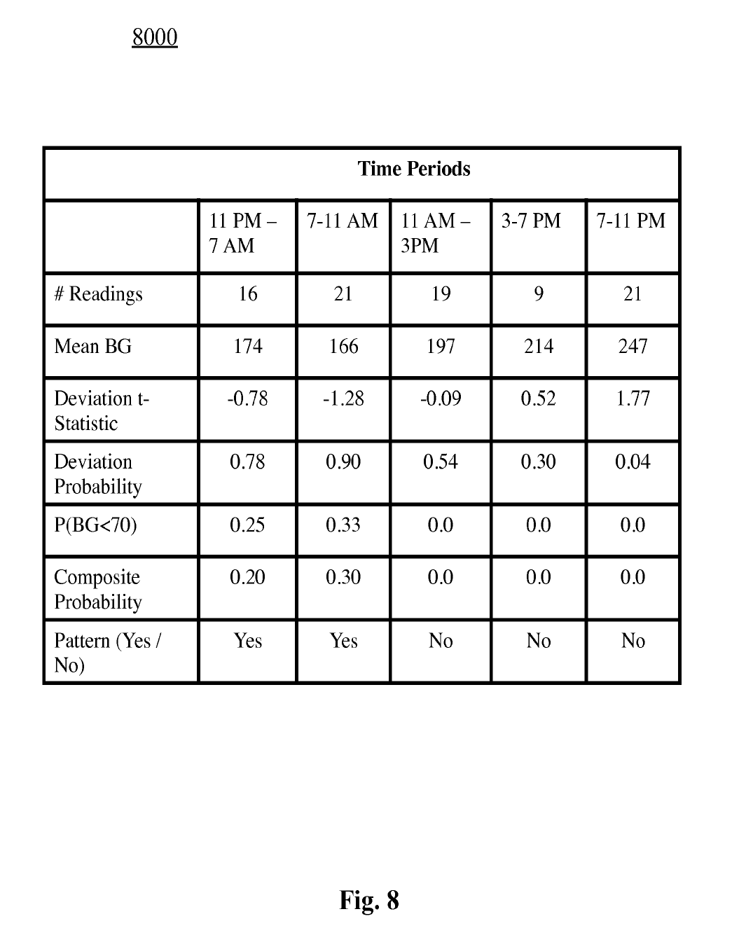

FIG. 8 is a table of exemplary statistical values identifying temporal patterns of hypoglycemia, illustrated by the identification of the time period from 11 pm to 11 am as a period wherein there is a pattern of hypoglycemia when the BG threshold parameter is set at 70 mg/dL and the composite probability threshold is set at 0.1.

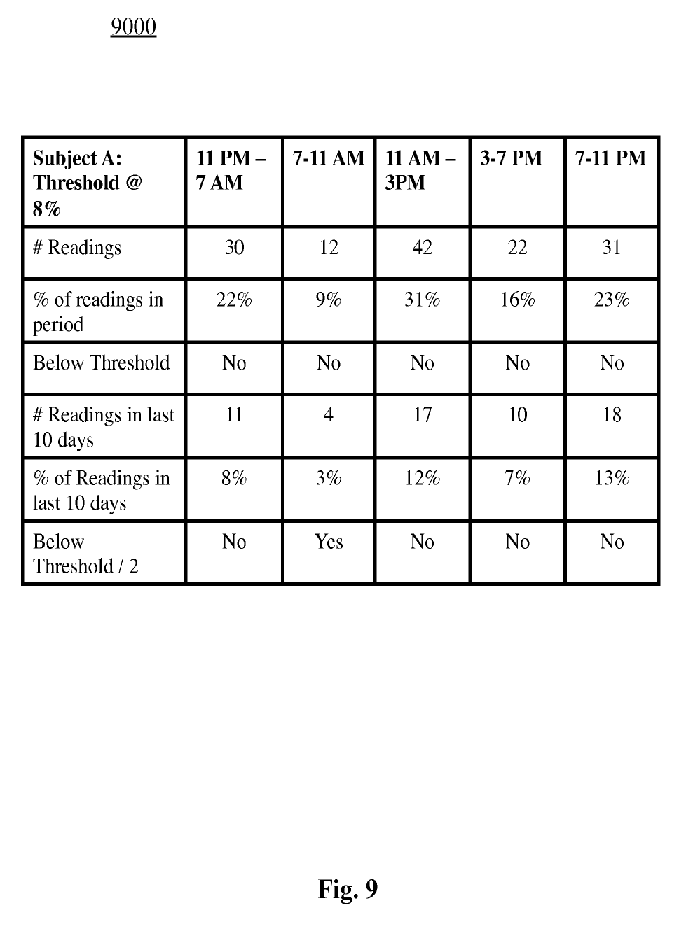

FIG. 9 is a table of exemplary statistical values identifying patterns of inadequate testing using Criterion C, where a time period is considered inadequate if it has less than a threshold percent of the total readings over the evaluable period (30 days in this case), or if it has less than half that threshold percent in the last 10 days. In this case, the percent of readings in morning time period (7-11 am) exceeds the threshold, but in the most recent 10 days the percentage is less than half the threshold percentage, therefore this is considered a period of inadequate testing.

FIG. 10 is a an exemplary embodiment of a diagram of glucose testing patterns, which comprise deleterious glucose patterns and periods of inadequate testing by time period that can be provided to the user. There are four deleterious glucose patterns identified (High in the morning, Low at mid-day, High in the evening and one other shown behind it). There is a pattern of inadequate testing shown at night.

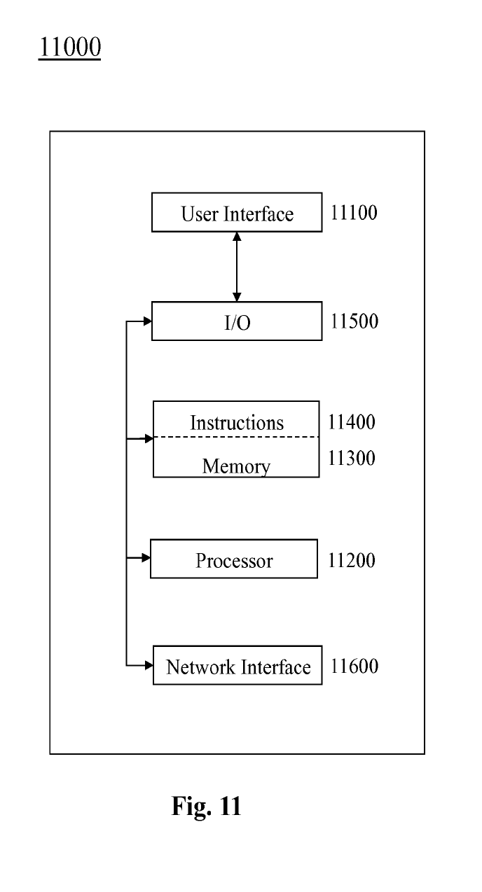

FIG. 11 is a block diagram of an exemplary embodiment of an information device 11000. Information device 11000 can comprise any of numerous circuits and/or components, such as for example, one or more network interfaces 11100, one or more processors 11200, one or more memories 11300 containing instructions 11400, one or more input/output (I/O) devices 11500, and/or one or more user interfaces 11600 coupled to I/O device 11500, etc.

In certain exemplary embodiments, via one or more user interfaces 11600, such as a graphical user interface, a user can view a rendering of information related to improved management of blood glucose testing and/or control.

### DEFINITIONS

When the following terms are used substantively herein, the accompanying definitions apply. These terms and definitions are presented without prejudice, and, consistent with the application, the right to redefine these terms during the prosecution of this application or any application claiming priority hereto is reserved. For the purpose of interpreting a claim of any patent that claims priority hereto, each definition (or redefined term if an original definition was amended during the prosecution of that patent), functions as a clear and unambiguous disavowal of the subject matter outside of that definition.
a—at least one.
activity—an action, act, step, and/or process or portion thereof
adapted to—made suitable or fit for a specific use or situation.
adequate percentage of tests—a sufficient rate or proportion per hundred of blood glucose evaluations to satisfy a predetermined condition.
and/or—either in conjunction with or in alternative to.
apparatus—an appliance or device for a particular purpose
associate—to join, connect together, and/or relate.
automatically—acting or operating in a manner essentially independent of external influence or control. For example, an automatic light switch can turn on upon “seeing” a person in its view, without the person manually operating the light switch.
blood glucose test—a measurement of the amount of a sugar called glucose in a sample of blood.
blood glucose testing patterns—a profile, density, or paucity of blood glucose testing over one or more periods of time.
can—is capable of in at least some embodiments.
comprising—including but not limited to.
configure—to make suitable or fit for a specific use or situation.
convert—to transform, adapt, and/or change.
count—a number representing the result of a process of counting.
couple—to join, connect, and/or link together.
daily frequency—a count of times something is done during a twenty four hour time period, or an average or median count of times something is done over a plurality of twenty four hour time periods.
daily time periods—defined intervals of time within a twenty four hour time period, possibly stratified over a number of days, weeks, or months.
data—distinct pieces of information, usually formatted in a special or predetermined way and/or organized to express concepts.
day—a 24 hour period of time, whether or not defined by one calendar day. For example 11:00 pm January 5^{th} to 11:00 pm January 6^{th} can comprise one day of time.
deleterious glucose patterns—a profile of blood glucose values, which profile is associated with one or more negative health conditions such as blood glucose variability, excessive high blood glucose, or excessive low blood glucose.
define—to establish the outline, form, or structure of
determine—to obtain, calculate, decide, deduce, and/or ascertain.
diabetic patient—a person having diabetes mellitus of any type, whether or not diagnosed.
device—a machine, manufacture, and/or collection thereof
during—at some point in the course of.
estimate—to calculate and/or determine approximately and/or tentatively.
evaluate—to determine the significance of, to assess.
generate—to create, produce, give rise to, and/or bring into existence.
glucose monitoring device—a device adapted to measure a sugar called glucose in a blood sample.
glucose variability—fluctuations in measurements of a sugar called glucose in blood over time.
haptic—involving the human sense of kinesthetic movement and/or the human sense of touch. Among the many potential haptic experiences are numerous sensations, body-positional differences in sensations, and time-based changes in sensations that are perceived at least partially in non-visual, non-audible, and non-olfactory manners, including the experiences of tactile touch (being touched), active touch, grasping, pressure, friction, traction, slip, stretch, force, torque, impact, puncture, vibration, motion, acceleration, jerk, pulse, orientation, limb position, gravity, texture, gap, recess, viscosity, pain, itch, moisture, temperature, thermal conductivity, and thermal capacity.
hyperglycemia—a condition characterized by blood glucose concentrations above a normal range.
hypoglycemia—a condition characterized by blood glucose concentrations below a normal range.
identify—to recognize or establish something.
inadequate—not sufficient.
indicate—to show the presence of a medical condition.
information device—any device capable of processing data and/or information, such as any general purpose and/or special purpose computer, such as a personal computer, workstation, server, minicomputer, mainframe, supercomputer, computer terminal, laptop, wearable computer, and/or Personal Digital Assistant (PDA), mobile terminal, Bluetooth device, communicator, “smart” phone (such as a Treo-like device), messaging service (e.g., Blackberry) receiver, pager, facsimile, cellular telephone, a traditional telephone, telephonic device, a programmed microprocessor or microcontroller and/or peripheral integrated circuit elements, an ASIC or other integrated circuit, a hardware electronic logic circuit such as a discrete element circuit, and/or a programmable logic device such as a PLD, PLA, FPGA, or PAL, or the like, etc. In general any device on which resides a finite state machine capable of implementing at least a portion of a method, structure, and/or or graphical user interface described herein may be used as an information device. An information device can comprise components such as one or more network interfaces, one or more processors, one or more memories containing instructions, and/or one or more input/output (I/O) devices, one or more user interfaces coupled to an I/O device, etc.
initiate—to start or begin.
input/output (I/O) device—any sensory-oriented input and/or output device, such as an audio, visual, electronic, signal processor, haptic, olfactory, and/or taste-oriented device, including, for example, a monitor, display, projector, overhead display, keyboard, keypad, mouse, trackball, joystick, gamepad, wheel, touchpad, touch panel, pointing device, microphone, speaker, video camera, camera, scanner, printer, haptic device, vibrator, tactile simulator, and/or tactile pad, potentially including a port to which an I/O device can be attached or connected.
instruction—an order or direction to do something.
insufficient blood glucose testing or insufficient blood glucose testing pattern—measurements of a sugar called glucose in blood that have not been taken often enough to satisfy one or more criterion established for frequency, profile, density, or paucity of such measurements.
machine instructions—directions adapted to cause a machine, such as an information device, to perform one or more particular activities, operations, or functions. The directions, which can sometimes form an entity called a “processor”, “kernel”, “operating system”, “program”, “application”, “utility”, “subroutine”, “script”, “macro”, “file”, “project”, “module”, “library”, “class”, and/or “object”, etc., can be embodied as machine code, source code, object code, compiled code, assembled code, interpretable code, and/or executable code, etc., in hardware, firmware, and/or software.
machine readable medium—a physical structure from which a machine can obtain data and/or information. Examples include a memory, punch cards, etc.
may—is allowed and/or permitted to, in at least some embodiments.
memory device—an apparatus capable of storing analog or digital information, such as instructions and/or data. Examples include a non-volatile memory, volatile memory, Random Access Memory, RAM, Read Only Memory, ROM, flash memory, magnetic media, a hard disk, a floppy disk, a magnetic tape, an optical media, an optical disk, a compact disk, a CD, a digital versatile disk, a DVD, and/or a raid array, etc. The memory device can be coupled to a processor and/or can store instructions adapted to be executed by processor, such as according to an embodiment disclosed herein.
message—a communication comprising information.
method—a process, procedure, and/or collection of related activities for accomplishing something.
network—a communicatively coupled plurality of nodes. A network can be and/or utilize any of a wide variety of sub-networks, such as a circuit switched, public-switched, packet switched, data, telephone, telecommunications, video distribution, cable, terrestrial, broadcast, satellite, broadband, corporate, global, national, regional, wide area, backbone, packet-switched TCP/IP, Fast Ethernet, Token Ring, public Internet, private, ATM, multi-domain, and/or multi-zone sub-network, one or more Internet service providers, and/or one or more information devices, such as a switch, router, and/or gateway not directly connected to a local area network, etc.
network interface—any device, system, or subsystem capable of coupling an information device to a network. For example, a network interface can be a telephone, cellular phone, cellular modem, telephone data modem, fax modem, wireless transceiver, Ethernet card, Ethernet cable, Bluetooth connector, cellular connector, radio-frequency connector, fibre-optic cable, transistor, cable modem, digital subscriber line interface, bridge, hub, router, or other similar device.
pattern—a combination of values forming a characteristic arrangement.
percent of total blood glucose readings—a rate or proportion per hundred of blood glucose evaluations.
perform—to carry out a method, activity, or task.
periods of insufficient blood glucose testing—a time interval determined, based upon one or more criterion, to not have enough testing of a patient's blood glucose levels.
plurality—the state of being plural and/or more than one.
predetermined—established in advance.
processor—a device and/or set of machine-readable instructions for performing one or more predetermined tasks. A processor can comprise any one or a combination of hardware, firmware, and/or software. A processor can utilize mechanical, pneumatic, hydraulic, electrical, magnetic, optical, informational, chemical, and/or biological principles, signals, and/or inputs to perform the task(s). In certain embodiments, a processor can act upon information by manipulating, analyzing, modifying, converting, transmitting the information for use by an executable procedure and/or an information device, and/or routing the information to an output device. A processor can function as a central processing unit, local controller, remote controller, parallel controller, and/or distributed controller, etc. Unless stated otherwise, the processor can be a general-purpose device, such as a microcontroller and/or a microprocessor, such the Pentium IV series of microprocessor manufactured by the Intel Corporation of Santa Clara, Calif. In certain embodiments, the processor can be dedicated purpose device, such as an Application Specific Integrated Circuit (ASIC) or a Field Programmable Gate Array (FPGA) that has been designed to implement in its hardware and/or firmware at least a part of an embodiment disclosed herein.
project—to calculate, estimate, or predict.
provide—to furnish, supply, give, and/or make available.
receive—to get as a signal, take, acquire, and/or obtain.
recommend—to suggest, praise, commend, and/or endorse.
render—to make perceptible to a human, for example as data, commands, text, graphics, audio, video, animation, and/or hyperlinks, etc., such as via any visual, audio, and/or haptic means, such as via a display, monitor, electric paper, ocular implant, cochlear implant, speaker, etc.
repeatedly—again and again; repetitively.
request—to express a desire for and/or ask for.
save—to store on a memory device.
set—a related plurality.
shorter than a length of—not having as long of a time duration compared to a predetermined time duration.
signal—information, such as machine instructions for activities and/or one or more letters, words, characters, symbols, signal flags, visual displays, and/or special sounds, etc. having prearranged meaning, encoded as automatically detectable variations in a physical variable, such as a pneumatic, hydraulic, acoustic, fluidic, mechanical, electrical, magnetic, optical, chemical, and/or biological variable, such as power, energy, pressure, flowrate, viscosity, density, torque, impact, force, frequency, phase, voltage, current, resistance, magnetomotive force, magnetic field intensity, magnetic field flux, magnetic flux density, reluctance, permeability, index of refraction, optical wavelength, polarization, reflectance, transmittance, phase shift, concentration, and/or temperature, etc. Depending on the context, a signal and/or the information encoded therein can be synchronous, asynchronous, hard real-time, soft real-time, non-real time, continuously generated, continuously varying, analog, discretely generated, discretely varying, quantized, digital, broadcast, multicast, unicast, transmitted, conveyed, received, continuously measured, discretely measured, processed, encoded, encrypted, multiplexed, modulated, spread, de-spread, demodulated, detected, de-multiplexed, decrypted, and/or decoded, etc.
store—to place, hold, and/or retain data, typically in a memory.
subsequent—after in time or placement.
substantially—to a great extent or degree.
system—a collection of mechanisms, devices, machines, articles of manufacture, processes, data, and/or instructions, the collection designed to perform one or more specific functions.
threshold—a predetermined level.
time—the system of those sequential relations that any event has to any other, as past, present, or future; indefinite and continuous duration regarded as that in which events succeed one another.
time period—a temporal interval having a defined duration.
transmit—to send as a signal, provide, furnish, and/or supply.
user interface—any device for rendering information to a user and/or requesting information from the user. A user interface includes at least one of textual, graphical, audio, video, animation, and/or haptic elements. A textual element can be provided, for example, by a printer, monitor, display, projector, etc. A graphical element can be provided, for example, via a monitor, display, projector, and/or visual indication device, such as a light, flag, beacon, etc. An audio element can be provided, for example, via a speaker, microphone, and/or other sound generating and/or receiving device. A video element or animation element can be provided, for example, via a monitor, display, projector, and/or other visual device. A haptic element can be provided, for example, via a very low frequency speaker, vibrator, tactile stimulator, tactile pad, simulator, keyboard, keypad, mouse, trackball, joystick, gamepad, wheel, touchpad, touch panel, pointing device, and/or other haptic device, etc. A user interface can include one or more textual elements such as, for example, one or more letters, number, symbols, etc. A user interface can include one or more graphical elements such as, for example, an image, photograph, drawing, icon, window, title bar, panel, sheet, tab, drawer, matrix, table, form, calendar, outline view, frame, dialog box, static text, text box, list, pick list, pop-up list, pull-down list, menu, tool bar, dock, check box, radio button, hyperlink, browser, button, control, palette, preview panel, color wheel, dial, slider, scroll bar, cursor, status bar, stepper, and/or progress indicator, etc. A textual and/or graphical element can be used for selecting, programming, adjusting, changing, specifying, etc. an appearance, background color, background style, border style, border thickness, foreground color, font, font style, font size, alignment, line spacing, indent, maximum data length, validation, query, cursor type, pointer type, autosizing, position, and/or dimension, etc. A user interface can include one or more audio elements such as, for example, a volume control, pitch control, speed control, voice selector, and/or one or more elements for controlling audio play, speed, pause, fast forward, reverse, etc. A user interface can include one or more video elements such as, for example, elements controlling video play, speed, pause, fast forward, reverse, zoom-in, zoom-out, rotate, and/or tilt, etc. A user interface can include one or more animation elements such as, for example, elements controlling animation play, pause, fast forward, reverse, zoom-in, zoom-out, rotate, tilt, color, intensity, speed, frequency, appearance, etc. A user interface can include one or more haptic elements such as, for example, elements utilizing tactile stimulus, force, pressure, vibration, motion, displacement, temperature, etc.
via—by way of and/or utilizing.
warn—to notify or make someone aware of potential danger or harm.

### Note

Still other substantially and specifically practical and useful embodiments will become readily apparent to those skilled in this art from reading the above-recited and/or herein-included detailed description and/or drawings of certain exemplary embodiments. It should be understood that numerous variations, modifications, and additional embodiments are possible, and accordingly, all such variations, modifications, and embodiments are to be regarded as being within the scope of this application.

Thus, regardless of the content of any portion (e.g., title, field, background, summary, description, abstract, drawing figure, etc.) of this application, unless clearly specified to the contrary, such as via explicit definition, assertion, or argument, with respect to any claim, whether of this application and/or any claim of any application claiming priority hereto, and whether originally presented or otherwise:
there is no requirement for the inclusion of any particular described or illustrated characteristic, function, activity, or element, any particular sequence of activities, or any particular interrelationship of elements;
no characteristic, function, activity, or element is “essential”;
any elements can be integrated, segregated, and/or duplicated;
any activity can be repeated, any activity can be performed by multiple entities, and/or any activity can be performed in multiple jurisdictions; and
any activity or element can be specifically excluded, the sequence of activities can vary, and/or the interrelationship of elements can vary.

Moreover, when any number or range is described herein, unless clearly stated otherwise, that number or range is approximate. When any range is described herein, unless clearly stated otherwise, that range includes all values therein and all subranges therein. For example, if a range of 1 to 10 is described, that range includes all values therebetween, such as for example, 1.1, 2.5, 3.335, 5, 6.179, 8.9999, etc., and includes all subranges therebetween, such as for example, 1 to 3.65, 2.8 to 8.14, 1.93 to 9, etc.

When any claim element is followed by a drawing element number, that drawing element number is exemplary and non-limiting on claim scope. No claim of this application is intended to invoke paragraph six of 35 USC 112 unless the precise phrase “means for” is followed by a gerund.

Any information in any material (e.g., a United States patent, United States patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such material is specifically not incorporated by reference herein.

Accordingly, every portion (e.g., title, field, background, summary, description, abstract, drawing figure, etc.) of this application, other than the claims themselves, is to be regarded as illustrative in nature, and not as restrictive, and the scope of subject matter protected by any patent that issues based on this application is defined only by the claims of that patent.

What is claimed is:

1. A method for identifying blood glucose patterns, comprising:
via a user interface, rendering a message in a first time period of a day that comprises information about at least:
one pattern of insufficient blood glucose testing in a second time period of said day, said second time period subsequent to said first time period; and
at least one pattern of deleterious blood glucose in a third time period of said day, said third time period subsequent to said second time period of said day;
wherein said message is based upon a determination that identifies daily periods with at least one of:
deleterious glucose patterns, said deleterious glucose patterns comprising at least one of an indication of hyperglycemia, hypoglycemia, and glucose variability; and
insufficient blood glucose testing during one or more time periods out of a plurality of time periods during said day.

2. The method of claim 1, wherein:
said determination of insufficient blood glucose testing comprises at least one of:
whether a percent of total readings in a first predetermined time period is below a first predetermined threshold; and
whether a count of readings in a second predetermined time period is below a second predetermined threshold

3. The method of claim 2, wherein:
insufficient blood glucose testing can also be identified if a percent of total readings in a third predetermined time period is below a third predetermined threshold, wherein said third predetermined time period is shorter than said first predetermined time period.

4. The method of claim 3, wherein:
said first predetermined time period is between approximately 14 days and approximately 60 days, and said third predetermined time period is between approximately 5 and approximately 30 days.

5. The method of claim 1, wherein:
said message is accompanied by at least one instruction or recommendation for a diabetic patient to test in a time period subsequent to said first time period.

6. The method of claim 1, wherein:
said determination of daily periods is initiated by an entry of a blood glucose reading.

7. A method for identifying blood glucose patterns, comprising:
in a first time period making a determination of at least one of:
daily periods with deleterious glucose patterns, said deleterious glucose patterns comprising at least one of an indication of hyperglycemia, hypoglycemia, and glucose variability; and
insufficient blood glucose testing during one or more time periods out of a plurality of time periods during a day;
saving an indication concerning at least one time period of insufficient blood glucose testing or deleterious glucose patterns adapted for rendering to a user in a second time period, said second time period subsequent to said first time period; and
via a user interface, rendering a message in said second time period of said day about said deleterious glucose patterns or insufficient blood glucose testing patterns occurring in one or more time periods of said day subsequent to said second time period.

8. The method of claim 7, wherein:
said message in said second time period of said day relates to at least one pattern of:
insufficient blood glucose testing in a third time period of said day, said third time period subsequent to said second time period; and
at least one pattern of deleterious blood glucose in a fourth time period of said day, said fourth time period subsequent to said third time period of said day.

9. The method of claim 7, wherein:
said determination of insufficient blood glucose testing comprises at least one of:
whether a percent of total readings in a first predetermined time period is below a first predetermined threshold; and
whether a count of readings in a second predetermined time period is below a second predetermined threshold.

10. The method of claim 9, wherein:
insufficient blood glucose testing can also be identified if a percent of total readings in a third predetermined time period is determined to be below a third predetermined threshold, and wherein said third predetermined time period is shorter than said first predetermined time period.

11. The method of claim 10, wherein:
said first predetermined time period is between approximately 14 days and approximately 60 days, and said third predetermined time period is between approximately 5 and approximately 30 days.

12. The method of claim 7, wherein:
said message is accompanied by at least one instruction or recommendation for a diabetic patient to test in a time period subsequent to said second time period.

13. The method of claim 7, wherein:
said determination of daily periods is initiated by an entry or acceptance of a blood glucose reading.

14. A machine-readable medium comprising machine-implementable instructions for activities comprising:
via a user interface, rendering a message in a first time period of a day about insufficient blood glucose testing in a second time period of said day, said second time period subsequent to said first time period, and about at least one pattern of deleterious blood glucose in a third time period of said day that is subsequent to said second time period of said day, wherein:
said message is based upon an evaluation of blood glucose readings stored in a memory device that determines daily periods with at least one of:
deleterious glucose patterns, said deleterious glucose patterns comprising at least one of an indication of hyperglycemia, hypoglycemia, and glucose variability; and
insufficient blood glucose testing during one or more time periods out of a plurality of time periods during said day.

15. The machine-readable medium of claim 14 wherein:
said message comprises information concerning:
at least one pattern of insufficient blood glucose testing in a time period of said day subsequent to said second time period; and
at least one pattern of deleterious blood glucose in said third time period of said day.

16. The machine-readable medium of claim 15 wherein:
said message is rendered responsive to a determination of at least one of:
a percentage of total readings in a first predetermined time period is below a first predetermined threshold; and
a count of readings in a second predetermined time period is below a second predetermined threshold.

17. The machine-readable medium of claim 16 wherein:
said message is rendered responsive to a determination that a percentage of total readings in a third predetermined time period is below a third predetermined threshold; and
said third predetermined time period is shorter than said first predetermined time period.

18. The machine-readable medium of claim 17 wherein:
said message is rendered responsive to a determination that said first predetermined time period is between approximately 14 days and approximately 60 days, and said third predetermined time period is between approximately 5 and approximately 30 days.

19. The machine-readable medium of claim 14 further comprising instructions for activities comprising:
rendering at least one instruction or recommendation, via said user interface, for a diabetic patient to test in a time period subsequent to said first time period.

20. The machine-readable medium of claim 14 further comprising instructions for activities comprising:
receiving a blood glucose reading through said user interface to initiate said determination of daily periods.

21. A system comprising:
a means for making a determination in a first time period of a day of at least one of:
daily periods with deleterious glucose patterns, said deleterious glucose patterns comprising at least one of an indication of hyperglycemia, hypoglycemia, and glucose variability; and
insufficient blood glucose testing by evaluating blood glucose readings stored in a memory device during one or more time periods out of a plurality of time periods during said day;
a means for storing on a memory device an indication concerning said determination such that information concerning said determination is renderable in a second time period that is subsequent to said first time period; and
a means for, via a user interface, rendering a message in said second time period about said determination in one or more time periods of said day subsequent to said first time period.

22. The system of claim 21, further comprising:
a means for rendering said message, via said user interface, in a second time period of said day when it relates at least one pattern of insufficient blood glucose testing in a subsequent third time period of said day, and at least one pattern of deleterious blood glucose in a fourth time period of said day that is subsequent to said third time period of said day.

23. The system of claim 22 further comprising:
a means for rendering said message, via said user interface, responsive to a determination that at least one of:
a percent of total readings in a first predetermined time period is below a first predetermined threshold; and
a count of readings in a second predetermined time period is below a second predetermined threshold.

24. The system of claim 23 further comprising:
a means for rendering said message, via said user interface, if a percent of total readings in a third predetermined time period is below a third predetermined threshold, wherein said third predetermined time period is shorter than said first predetermined time period.

25. The system of claim 24 further comprising:
a means for rendering said message, via said user interface, if said first predetermined time period is between approximately 14 days and approximately 60 days, and said third predetermined time period is between approximately 5 and approximately 30 days.

26. The system of claim 21, further comprising:
a means for rendering at least one instruction or recommendation, via said user interface, for a diabetic patient to test in a time period subsequent to said second time period.

27. The system of claim 21, further comprising:
a means for entering or accepting a blood glucose reading through said user interface to initiate said determination.

28. A method for identifying blood glucose patterns, comprising:
via a user interface, rendering a message in a first time period of a day about at least one pattern of:
insufficient blood glucose testing; and
deleterious blood glucose, said deleterious glucose patterns comprising at least one of an indication of hyperglycemia, hypoglycemia, and glucose variability;
wherein said message communicates, in one display, for every time period of said day, an identification of any patterns of insufficient blood glucose testing or deleterious blood glucose; and
wherein said message is based upon a determination that identifies said at least one pattern during one or more time periods out of a plurality of time periods during said day.

29. A system comprising:
a means for rendering a message, via a user interface, in a first time period of a day about at least one pattern of:
insufficient blood glucose testing; and
deleterious blood glucose, said deleterious glucose patterns comprising at least one of an indication of hyperglycemia, hypoglycemia, and glucose variability;
wherein said message communicates, in one display, for every time period of said day, an identification of any patterns of insufficient blood glucose testing or deleterious blood glucose; and
wherein said message is based upon a determination of said at least one pattern during one or more time periods out of a plurality of time periods during said day.

30. A method for identifying blood glucose patterns, comprising:
via a user interface, rendering a message in a first time period of a day that comprises information about at least one insufficient blood glucose testing pattern in one or more time periods of said day subsequent to said first time period;
wherein said message is based upon a determination that identifies at least one of:
daily periods with deleterious glucose patterns, said deleterious glucose patterns comprising at least one of an indication of hyperglycemia, hypoglycemia, and glucose variability; and
insufficient blood glucose testing during one or more time periods out of a plurality of time periods during said day; and
wherein said insufficient blood glucose testing is determined by criteria comprising whether a count of readings in a predetermined time period is below a predetermined threshold.

31. A method for identifying blood glucose patterns, comprising:
via a user interface, rendering a message in a first time period of a day that comprises information about at least one insufficient blood glucose testing pattern in one or more time periods of said day subsequent to said first time period;
wherein said message is based upon a determination that identifies daily periods with at least one of:
deleterious glucose patterns, said deleterious glucose patterns comprising at least one of an indication of hyperglycemia, hypoglycemia, and glucose variability; and
insufficient blood glucose testing during one or more time periods out of a plurality of time periods during said day, and
wherein said insufficient blood glucose testing is determined by whether a percent of total readings in a first predetermined time period is below a first predetermined threshold, or whether a percent of total readings in a second predetermined time period is below a second predetermined threshold, wherein said second predetermined time period is shorter than said first predetermined time period.

32. A system comprising;
a means for rendering a message, via a user interface, in a first time period of a day that comprises information about at least one insufficient blood glucose testing pattern in one or more time periods of said day subsequent to said first time period,
wherein said message is based upon a determination that identifies daily periods with at least one of:
deleterious glucose patterns, said deleterious glucose patterns comprising at least one of an indication of hyperglycemia, hypoglycemia, and glucose variability; and
insufficient blood glucose testing patterns during one or more time periods out of a plurality of time periods during said day; and
wherein said insufficient blood glucose testing is determined based upon criteria comprising at least one of whether a count of readings in a predetermined time period is below a predetermined threshold.

33. A system comprising:
a means for rendering a message, via a user interface, in a first time period of a day that comprises information about at least one insufficient blood glucose testing pattern in one or more time periods of said day subsequent to said first time period;
wherein said message is based upon a determination that identifies daily periods with at least one of:
deleterious glucose patterns, said deleterious glucose patterns comprising at least one of an indication of hyperglycemia, hypoglycemia, and glucose variability; and
insufficient blood glucose testing patterns during one or more time periods out of a plurality of time periods during said day; and
wherein said insufficient blood glucose testing is determined by whether a percent of total readings in a first predetermined time period is below a first predetermined threshold, or whether a percent of total readings in a second predetermined time period is below a second predetermined threshold, and wherein said second predetermined time period is shorter than said first predetermined time period.
